SEQUENCE LISTING (10 sequences)

SEQ ID NO: 1 - DNA, 2735 bp, Homo sapiens
  CD86 Variant 1
agtcattgccgaggaaggcttgcacagggtgaaagctttgcttctctgctgctgtaacagggactagcacagacacacggatgagtggggtcatttccagatattaggtcacagcagaagcagccaaaatggatccccagtgcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaatacctcctccagatcagattcttctcttaatttcatagattgtgttttttttttaaatagacctctcaatttctggaaaactgccttttatctgcccagaattctaagctggtgccccactgaattttgtgtacctgtgactaaacaactacctcctcagtctgggtgggacttatgtatttatgaccttatagtgttaatatcttgaaacatagagatctatgtactgtaatagtgtgattactatgctctagagaaaagtctacccctgctaaggagttctcatccctctgtcagggtcagtaaggaaaacggtggcctagggtacaggcaacaatgagcagaccaacctaaatttggggaaattaggagaggcagagatagaacctggagccacttctatctgggctgttgctaatattgaggaggcttgccccacccaacaagccatagtggagagaactgaataaacaggaaaatgccagagcttgtgaaccctgtttctcttgaagaactgactagtgagatggcctggggaagctgtgaaagaaccaaaagagatcacaatactcaaaagagagagagagagaaaaaagagagatcttgatccacagaaatacatgaaatgtctggtctgtccaccccatcaacaagtcttgaaacaagcaacagatggatagtctgtccaaatggacataagacagacagcagtttccctggtggtcagggaggggttttggtgatacccaagttattgggatgtcatcttcctggaagcagagctggggagggagagccatcaccttgataatgggatgaatggaaggaggcttaggactttccactcctggctgagagaggaagagctgcaacggaattaggaagaccaagacacagatcacccggggcttacttagcctacagatgtcctacgggaacgtgggctggcccagcatagggctagcaaatttgagttggatgattgtttttgctcaaggcaaccagaggaaacttgcatacagagacagatatactgggagaaatgactttgaaaacctggctctaaggtgggatcactaagggatggggcagtctctgcccaaacataaagagaactctggggagcctgagccacaaaaatgttcctttattttatgtaaaccctcaagggttatagactgccatgctagacaagcttgtccatgtaatattcccatgtttttaccctgcccctgccttgattagactcctagcacctggctagtttctaacatgttttgtgcagcacagtttttaataaatgcttgttacattcatttaaaaaaaaaaaaaa

SEQ ID NO: 2 - protein, 323 aa, Homo sapiens
  CD86 Variant 1
MGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 3 - DNA, 2790 bp, Homo sapiens
  CD86 Variant 2
ccctttctgtatttgagttctaccgtcagtcctggcattatttctctctctacaaggagccttaggaggtacggggagctcgcaaatactccttttggtttattcttaccaccttgcttctgtgttccttgggaatgctgctgtgcttatgcatctggtctctttttggagctacagtggacaggcatttgtgacagcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaatacctcctccagatcagattcttctcttaatttcatagattgtgttttttttttaaatagacctctcaatttctggaaaactgccttttatctgcccagaattctaagctggtgccccactgaattttgtgtacctgtgactaaacaactacctcctcagtctgggtgggacttatgtatttatgaccttatagtgttaatatcttgaaacatagagatctatgtactgtaatagtgtgattactatgctctagagaaaagtctacccctgctaaggagttctcatccctctgtcagggtcagtaaggaaaacggtggcctagggtacaggcaacaatgagcagaccaacctaaatttggggaaattaggagaggcagagatagaacctggagccacttctatctgggctgttgctaatattgaggaggcttgccccacccaacaagccatagtggagagaactgaataaacaggaaaatgccagagcttgtgaaccctgtttctcttgaagaactgactagtgagatggcctggggaagctgtgaaagaaccaaaagagatcacaatactcaaaagagagagagagagaaaaaagagagatcttgatccacagaaatacatgaaatgtctggtctgtccaccccatcaacaagtcttgaaacaagcaacagatggatagtctgtccaaatggacataagacagacagcagtttccctggtggtcagggaggggttttggtgatacccaagttattgggatgtcatcttcctggaagcagagctggggagggagagccatcaccttgataatgggatgaatggaaggaggcttaggactttccactcctggctgagagaggaagagctgcaacggaattaggaagaccaagacacagatcacccggggcttacttagcctacagatgtcctacgggaacgtgggctggcccagcatagggctagcaaatttgagttggatgattgtttttgctcaaggcaaccagaggaaacttgcatacagagacagatatactgggagaaatgactttgaaaacctggctctaaggtgggatcactaagggatggggcagtctctgcccaaacataaagagaactctggggagcctgagccacaaaaatgttcctttattttatgtaaaccctcaagggttatagactgccatgctagacaagcttgtccatgtaatattcccatgtttttaccctgcccctgccttgattagactcctagcacctggctagtttctaacatgttttgtgcagcacagtttttaataaatgcttgttacattcatttaaaaaaaaaaaaaa

SEQ ID NO: 4 - DNA, 2790 bp, Homo sapiens; identical to SEQ ID NO: 3
  CD86 Variant 2

SEQ ID NO: 5 - DNA, 2646 bp, Homo sapiens
  CD86 Variant 3
ccctttctgtatttgagttctaccgtcagtcctggcattatttctctctctacaaggagccttaggaggtacggggagctcgcaaatactccttttggtttattcttaccaccttgcttctgtgttccttgggaatgctgctgtgcttatgcatctggtctctttttggagctacagtggacaggcatttgtgacagcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctataggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaatacctcctccagatcagattcttctcttaatttcatagattgtgttttttttttaaatagacctctcaatttctggaaaactgccttttatctgcccagaattctaagctggtgccccactgaattttgtgtacctgtgactaaacaactacctcctcagtctgggtgggacttatgtatttatgaccttatagtgttaatatcttgaaacatagagatctatgtactgtaatagtgtgattactatgctctagagaaaagtctacccctgctaaggagttctcatccctctgtcagggtcagtaaggaaaacggtggcctagggtacaggcaacaatgagcagaccaacctaaatttggggaaattaggagaggcagagatagaacctggagccacttctatctgggctgttgctaatattgaggaggcttgccccacccaacaagccatagtggagagaactgaataaacaggaaaatgccagagcttgtgaaccctgtttctcttgaagaactgactagtgagatggcctggggaagctgtgaaagaaccaaaagagatcacaatactcaaaagagagagagagagaaaaaagagagatcttgatccacagaaatacatgaaatgtctggtctgtccaccccatcaacaagtcttgaaacaagcaacagatggatagtctgtccaaatggacataagacagacagcagtttccctggtggtcagggaggggttttggtgatacccaagttattgggatgtcatcttcctggaagcagagctggggagggagagccatcaccttgataatgggatgaatggaaggaggcttaggactttccactcctggctgagagaggaagagctgcaacggaattaggaagaccaagacacagatcacccggggcttacttagcctacagatgtcctacgggaacgtgggctggcccagcatagggctagcaaatttgagttggatgattgtttttgctcaaggcaaccagaggaaacttgcatacagagacagatatactgggagaaatgactttgaaaacctggctctaaggtgggatcactaagggatggggcagtctctgcccaaacataaagagaactctggggagcctgagccacaaaaatgttcctttattttatgtaaaccctcaagggttatagactgccatgctagacaagcttgtccatgtaatattcccatgtttttaccctgcccctgccttgattagactcctagcacctggctagtttctaacatgttttgtgcagcacagtttttaataaatgcttgttacattcatttaaaaaaaaaaaaaa

SEQ ID NO: 6 - protein, 275 aa, Homo sapiens
  CD86 Variant 3
MGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIGTNTMEREESEQTKKREKIHIPERSDEAQRVFKSSKTSSCDKSDTCF

SEQ ID NO: 7 - DNA, 2399 bp, Homo sapiens
  CD86 Variant 4
agtcattgccgaggaaggcttgcacagggtgaaagctttgcttctctgctgctgtaacagggactagcacagacacacggatgagtggggtcatttccagatattaggtcacagcagaagcagccaaaatggatccccagtgcactatgggactgagtaacattctctttgtgatggccttcctgctctctgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaatacctcctccagatcagattcttctcttaatttcatagattgtgttttttttttaaatagacctctcaatttctggaaaactgccttttatctgcccagaattctaagctggtgccccactgaattttgtgtacctgtgactaaacaactacctcctcagtctgggtgggacttatgtatttatgaccttatagtgttaatatcttgaaacatagagatctatgtactgtaatagtgtgattactatgctctagagaaaagtctacccctgctaaggagttctcatccctctgtcagggtcagtaaggaaaacggtggcctagggtacaggcaacaatgagcagaccaacctaaatttggggaaattaggagaggcagagatagaacctggagccacttctatctgggctgttgctaatattgaggaggcttgccccacccaacaagccatagtggagagaactgaataaacaggaaaatgccagagcttgtgaaccctgtttctcttgaagaactgactagtgagatggcctggggaagctgtgaaagaaccaaaagagatcacaatactcaaaagagagagagagagaaaaaagagagatcttgatccacagaaatacatgaaatgtctggtctgtccaccccatcaacaagtcttgaaacaagcaacagatggatagtctgtccaaatggacataagacagacagcagtttccctggtggtcagggaggggttttggtgatacccaagttattgggatgtcatcttcctggaagcagagctggggagggagagccatcaccttgataatgggatgaatggaaggaggcttaggactttccactcctggctgagagaggaagagctgcaacggaattaggaagaccaagacacagatcacccggggcttacttagcctacagatgtcctacgggaacgtgggctggcccagcatagggctagcaaatttgagttggatgattgtttttgctcaaggcaaccagaggaaacttgcatacagagacagatatactgggagaaatgactttgaaaacctggctctaaggtgggatcactaagggatggggcagtctctgcccaaacataaagagaactctggggagcctgagccacaaaaatgttcctttattttatgtaaaccctcaagggttatagactgccatgctagacaagcttgtccatgtaatattcccatgtttttaccctgcccctgccttgattagactcctagcacctggctagtttctaacatgttttgtgcagcacagtttttaataaatgcttgttacattcatttaaaaaaaaaaaaaa

SEQ ID NO: 8 - protein, 217 aa, Homo sapiens
  CD86 Variant 4
MDPQCTMGLSNILFVMAFLLSANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 9 - DNA, 2685 bp, Homo sapiens
  CD86 Variant 5
agtcattgccgaggaaggcttgcacagggtgaaagctttgcttctctgctgctgtaacagggactagcacagacacacggatgagtggggtcatttccagatattaggtcacagcagaagcagccaaaatggatccccagtggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaatacctcctccagatcagattcttctcttaatttcatagattgtgttttttttttaaatagacctctcaatttctggaaaactgccttttatctgcccagaattctaagctggtgccccactgaattttgtgtacctgtgactaaacaactacctcctcagtctgggtgggacttatgtatttatgaccttatagtgttaatatcttgaaacatagagatctatgtactgtaatagtgtgattactatgctctagagaaaagtctacccctgctaaggagttctcatccctctgtcagggtcagtaaggaaaacggtggcctagggtacaggcaacaatgagcagaccaacctaaatttggggaaattaggagaggcagagatagaacctggagccacttctatctgggctgttgctaatattgaggaggcttgccccacccaacaagccatagtggagagaactgaataaacaggaaaatgccagagcttgtgaaccctgtttctcttgaagaactgactagtgagatggcctggggaagctgtgaaagaaccaaaagagatcacaatactcaaaagagagagagagagaaaaaagagagatcttgatccacagaaatacatgaaatgtctggtctgtccaccccatcaacaagtcttgaaacaagcaacagatggatagtctgtccaaatggacataagacagacagcagtttccctggtggtcagggaggggttttggtgatacccaagttattgggatgtcatcttcctggaagcagagctggggagggagagccatcaccttgataatgggatgaatggaaggaggcttaggactttccactcctggctgagagaggaagagctgcaacggaattaggaagaccaagacacagatcacccggggcttacttagcctacagatgtcctacgggaacgtgggctggcccagcatagggctagcaaatttgagttggatgattgtttttgctcaaggcaaccagaggaaacttgcatacagagacagatatactgggagaaatgactttgaaaacctggctctaaggtgggatcactaagggatggggcagtctctgcccaaacataaagagaactctggggagcctgagccacaaaaatgttcctttattttatgtaaaccctcaagggttatagactgccatgctagacaagcttgtccatgtaatattcccatgtttttaccctgcccctgccttgattagactcctagcacctggctagtttctaacatgttttgtgcagcacagtttttaataaatgcttgttacattcatttaaaaaaaaaaaaaa

SEQ ID NO: 10 - protein, 247 aa, Homo sapiens
  CD86 Variant 5
MGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

(19) United States
(12) Patent Application Publication
(10) Pub. No.: US 2016/0130659 A1
(43) Pub. Date: May 12, 2016

(54) COMPOSITIONS AND METHODS FOR TREATING CANCER

(71) Applicant: MERCK SHARP & DOHME CORP., Rahway, NJ (US)

(72) Inventors: Kenzie MacIsaac, Brookline, MA (US); Manfred Kraus, Brookline, MA (US); Peter Richard Strack, Reading, MA (US); Kumiko Nagashima, Belmont, MA (US); John Michael Ellis, Needham Heights, MA (US)

(73) Assignee: Merck Sharp & Dohme Corp., Rahway, NJ (US)

(21) Appl. No.: 14/894,705

(22) PCT Filed: Jun. 3, 2014
(86) PCT No.: PCT/US2014/040586

§ 371 Date: Nov. 30, 2015

Related U.S. Application Data
(60) Provisional application No. 61/831,344, filed on Jun. 5, 2013.

(51) Int. Cl. C12Q 1/68; C07K 16/28; G01N 33/574

(52) U.S. Cl. CPC C12Q 1/6886; G01N 33/57426; C07K 16/2827; C12Q 2600/106; C12Q 2600/158; G01N 2333/70532

(57) ABSTRACT

The instant invention relates to methods for the treatment of B-cell lymphomas and leukemia by administering a SYK inhibitor. In another embodiment, the invention relates to a method for treating a patient diagnosed with a B-cell lymphoma or leukemia, comprising administering a SYK inhibitor, wherein the B-cells of said patient to be treated are characterized by elevated expression levels of CD86.

### FIELD OF THE INVENTION

The present invention relates generally to the identification of a biomarker whose expression level is useful for predicting a patient's response to treatment with an anti-proliferative agent, in particular a SYK inhibitor. The expression level of the biomarker can be used to predict a patient presenting with a cancerous condition that is mediated by inhibition of apoptosis and who is likely to respond to treatment with a SYK inhibitor.

### BACKGROUND OF THE INVENTION

Spleen Tyrosine Kinase (SYK) is a protein tyrosine kinase which has been described as a key mediator of immunoreceptor signaling in a host of inflammatory cells including mast cells, B-cells, macrophages and neutrophils. These immunoreceptors, including Fc receptors and the B-cell receptor, are important for both allergic diseases and antibody-mediated autoimmune diseases and thus, pharmacologically interfering with SYK could conceivably treat these disorders.

Studies using cells from mice deficient in the Spleen Tyrosine Kinase (SYK) have demonstrated a non-redundant role of this kinase in B cell function. The deficiency in SYK is characterized by a block in B cell development (M. Turner et al., 1995, Nature, 379:298-302 and Cheng et al., 1995, Nature, 378: 303-306). These studies, along with studies on mature B cells deficient in SYK (Kurasaki et al., 2000, Immunol. Rev., 176:19-29), demonstrate that SYK is required for the differentiation and activation of B cells.

Diffuse large B-cell lymphomas (DLBCL), the most common subtype of B-cell non-Hodgkin lymphomas (B-NHL), is a heterogeneous disease with variability in clinical outcome, genetic features, and cells of origin. Attempts have been made to segment DLBCL patients based on their molecular expression profiles to predict patient outcomes with limited success. For example, Staudt et al. (2000, Nature, 403(6769):503-511) used gene expression to classify DLBCL patients based on B-cell differentiation and found that patients with germinal centre B-like DLBCL had a significantly better overall survival rate than patients with activated B-like DLBCL. Similarly, Shipp et al. (2005, Blood, 105(5):1851-1861) found three subsets of DLBCL patients described as oxidative phosphorylation, B-cell receptor/proliferation, and host response (HR), each having a discrete set of histological features that define tumor microenvironment and host inflammatory response as defining features of DLBCL. However, none of the identified subgroups have been correlated with a therapeutic or treatment protocol.

It has been suggested that many B-cell lymphomas depend on B-cell receptor (BCR)-mediated survival signals. It has also been suggested that SYK may be part of the mechanism by which this signaling is regulated by amplifying BCR signaling (Chen et al., 2008, Blood, 111(4):2230-2237). As such, disrupting BCR-induced signaling by inhibiting SYK, such as with an oral inhibitor of SYK, is an attractive therapeutic approach for treating B-cell non-Hodgkin lymphomas (B-NHL), which also includes follicular lymphoma (FL), and other non-Hodgkin lymphomas (NHL), such as, mantle cell lymphoma (MCL), marginal zone lymphoma (MZL), mucosa-associated lymphoid tissue lymphoma, lyphoplasmacytic lymphomas, and small lymphocytic leukemia/chronic lymphocytic leukemia (SLL/CLL) (Friedberg et al., 2010, Blood, 115(13):2578-2585). It has also been found that while SYK is activated in less than half (44%) of human DLBCL cell lines, of those in which SYK was activated, more than half were sensitive to a SYK inhibitor (Cheng et al., 2011, Blood, 118(24):6342-6352).

Thus, there is a need for biomarkers that can be used to predict which patients are amenable to treatment with specific therapies, particularly for patients who are non-responsive or who are likely to become refractive to first line therapies. It is, therefore, an object of this invention to provide a predictive biomarker to identify patients likely to respond to treatment with a SYK inhibitor.

### SUMMARY OF THE INVENTION

The instant invention relates generally to the identification of a predictive biomarker whose expression level is useful for evaluating and classifying patients for treatment with a SYK inhibitor. In one embodiment of the invention the predictive biomarker, CD86, is used to identify patients likely to respond to treatment with a SYK inhibitor. In another embodiment, the invention is a method for treating a patient diagnosed with a B-NHL or leukemia with a SYK inhibitor, wherein the cancer cells of said patient are characterized by high expression of CD86. In still another embodiment, the invention is a method for treating a B-NHL or leukemia patient who is sensitive or predicted to be sensitive to treatment with a SYK inhibitor, wherein the cancer cells of said patient are characterized by a level of expression of CD86 that is above that of a reference value. In another embodiment, the invention is a method to identify SYK inhibitors for use in treating a B-NHL or leukemia. In yet another embodiment, the invention is a kit for identifying patients likely to respond to treatment with a SYK inhibitor comprising reagents reacting to CD86.

### BRIEF DESCRIPTION OF THE DRAWINGS

FIG. 1 is a graphic that illustrates that the SYK signature score is correlated with SYK-1 sensitivity in diffuse large B-cell lymphoma (DLBCL) cell lines.

FIG. 2 is a graphic that illustrates the relationship between SYK sensitivity and the signature score in an independent panel of B-cell lines. The signature score represents the average log expression of all 46 genes in the signature.

FIG. 3 is a graphic that illustrates the receiver operating characteristic (ROC) curve for the predictive SYK signature applied to forty six B-cell lines treated with SYK-1. The area under the ROC curve is 0.83.

FIG. 4 is a graphic that illustrates the relationship between CD86 mRNA expression and SYK-1 sensitivity in the seventeen cell line DLBCL panel.

FIG. 5 is a graphic that illustrates that DLBCL cell lines expressing high levels of CD86 are sensitive to SYK-1.

FIG. 6A is a depiction of the tumor biopsy samples used to score CD86 levels on a tissue microarray. FIG. 6B is a graphical representation of the frequency of CD86 positive and negative samples.

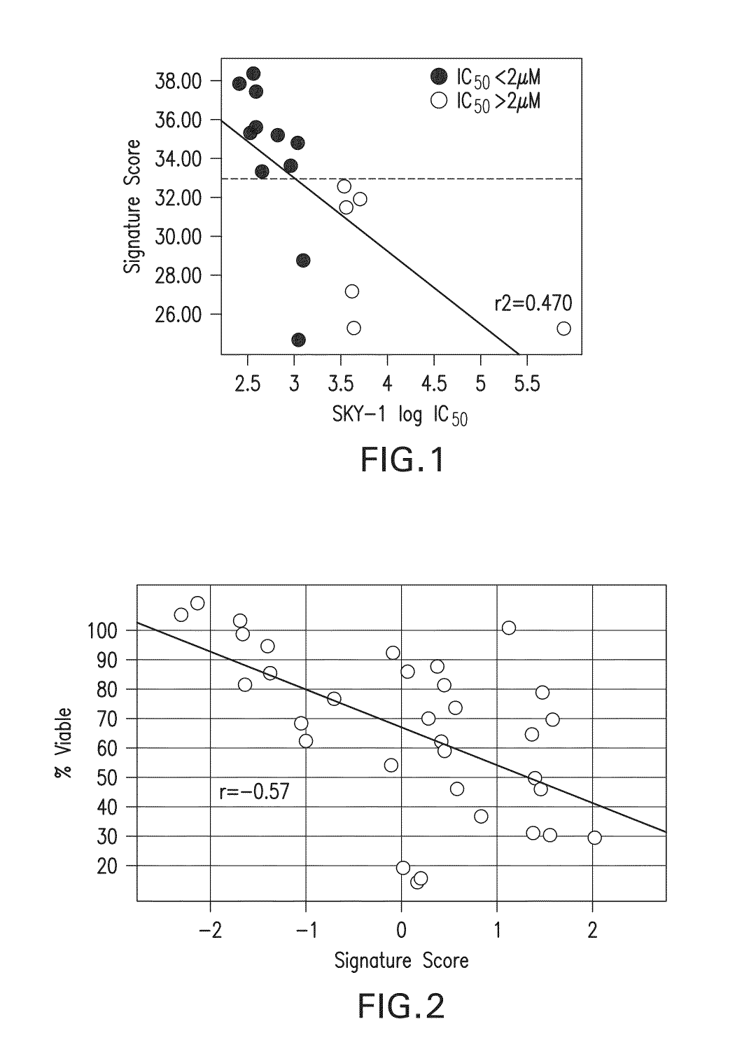

### DETAILED DESCRIPTION OF THE INVENTION

The invention herein relates to methods for treating a B-cell non-Hodgkin lymphoma (B-NHL) or leukemia patient with a SYK inhibitor. In another embodiment, the invention relates to a predictive biomarker, CD86, whose expression is sensitive to SYK inhibition. In still another embodiment, the invention relates to a method for treating a patient diagnosed with B-NHL or leukemia, in need of treatment thereof, with a SYK inhibitor, wherein the B-cells of said patient are characterized by high or elevated expression of CD86. In yet another embodiment, the invention is a method for treating a B-NHL or leukemia patient who is sensitive or predicted to be sensitive to treatment with a SYK inhibitor, wherein the B-cells of said patient are characterized by a level of expression of CD86 that is above that of a reference value. In another embodiment, the invention is a method to identify SYK inhibitors for use in treating a B-NHL or leukemia. In yet another embodiment, the invention is a kit for identifying patients likely to respond to treatment with a SYK inhibitor.

Therefore, the present invention provides a method for treating a patient diagnosed with a B-cell lymphoma or leukemia with a SYK inhibitor comprising the steps of:

(a) selecting a patient for treatment with a SYK inhibitor, wherein a malignant B-cell containing biological sample of said patient has elevated CD86 expression; and (b) administering a therapeutically effective amount of the SYK inhibitor to the selected patient.

In one embodiment, the method comprises the steps of: (a) measuring the expression level of CD86 in a malignant B-cell containing biological sample obtained from said patient;

(b) determining whether the CD86 expression level in said patient sample is above or below the level of a control sample;

(c) selecting said patient for treatment with a SYK inhibitor, wherein the level of CD86 expression in said patient sample is at or above that of the control sample; and

(d) administering a therapeutically effective amount of the SYK inhibitor to the selected patient.

In another embodiment, a malignant B-cell containing control sample is obtained from one or more subjects diagnosed with B-cell lymphoma or leukemia, but do not respond to treatment with a SYK inhibitor.

In another aspect, the invention provides a method for treating a patient diagnosed with a B-cell lymphoma or leukemia with a SYK inhibitor comprising:

(a) measuring the gene expression level of CD86 in a biological sample comprising malignant B-cells obtained from said patient and in a control sample;

(b) determining whether the CD86 gene expression level in said patient sample is above or below the level of that in said control sample;

(c) selecting said patient for treatment with a SYK inhibitor, wherein the level of the CD86 gene expression in said patient sample is at or above that of the control sample; and

(d) administering a SYK inhibitor to the selected patient.

In a further aspect, the invention provides a method for treating a B-cell lymphoma or leukemia patient sensitive to treatment with a SYK inhibitor comprising:

(a) measuring the gene expression level of CD86 in a biological sample comprising malignant B-cells obtained from said patient and in a control sample;

(b) determining whether the CD86 gene expression level in said patient sample is above or below the level of that in said control sample;

(c) identifying said sensitive patient for treatment with a SYK inhibitor, wherein the level of CD86 from said patient sample is at or above that of the control sample; and

(d) administering a SYK inhibitor to the sensitive patient.

In a further aspect, the invention provides a method for treating a B-cell lymphoma or leukemia patient predicted to be sensitive to treatment with a SYK inhibitor comprising:

(a) measuring the gene expression level of CD86 in a malignant B-cell containing biological sample obtained from said patient and in a control sample;

(b) determining whether the CD86 gene expression level in said patient sample is above or below the level of that in said control sample;

(c) identifying said patient for treatment with a SYK inhibitor, wherein the level of CD86 from said patient sample is at or above that of the control sample; and

(d) administering a SYK inhibitor to the patient.

The invention also provides a method for treating a patient diagnosed with a B-cell lymphoma or leukemia, in which a biological sample of the patient comprising malignant B-cells has elevated expression levels of CD86, comprising the step of: administering a therapeutically effective amount of a SYK inhibitor to the patient. Another embodiment of the invention is a SYK inhibitor for use in the treatment of patients with B-cell lymphoma or leukemia and elevated expression levels of CD86 in malignant B-cells.

The invention further provides a method for treating a B-cell lymphoma or leukemia patient, comprising the step of administering a therapeutically effective amount of a SYK inhibitor to the patient, wherein a biological sample of said patient comprising malignant B-cells is characterized by elevated expression of CD86.

In one embodiment of the above methods, the elevated expression levels of CD86 is in comparison to the CD86 expression level of a malignant B-cell containing biological sample of one or more subjects diagnosed with B-cell lymphoma or leukemia but do not respond to treatment with a SYK inhibitor. In one embodiment of the above methods, the elevated expression levels of CD86 is in comparison to a reference derived from a malignant B-cell containing biological sample of one or more subjects diagnosed with B-cell lymphoma or leukemia but do not respond to treatment with a SYK inhibitor.

In another embodiment of the above methods, the mRNA or protein expression level of CD86 is measured.

In a further embodiment of the above methods, the B-cell lymphoma or leukemia is selected from the group consisting of acute leukemia, chronic lymphatic leukemia, chronic myelocytic leukemia, and non-Hodgkin's lymphoma. In yet a further embodiment, the patient is diagnosed with diffuse large B-cell lymphoma.

In yet a further aspect, the invention provides a kit to identify a B-cell lymphoma or leukemia patient predicted to be sensitive to treatment with a SYK inhibitor comprising a detection agent capable of detecting the expression product of CD86 in a biological test sample. In one embodiment, the expression product is mRNA transcript of CD86 or the CD86 protein.

The term “B-cell lymphoma” as used herein refers to any of the various non-Hodgkin lymphomas characterized by tumors expressing one or more B-cell antigens or by malignant transformation of the B lymphocytes. Non-Hodgkin lymphomas as a group are heterogeneous with respect to malignant cell lineage, clinical course, prognosis, and therapy. The only common feature for this large group of lymphomas is the absence of Reed-Sternberg cells, which are characteristic of Hodgkin disease. Examples of this group of lymphomas includes, but is not limited to, diffuse lymphoma, follicular lymphoma, large cell lymphoma, diffuse large B-cell lymphoma, mantle cell lymphoma, marginal zone lymphoma, mucosa-associated lymphoid tissue lymphoma, lyphoplasmacytic lymphoma, small cell lymphoma, small cleaved/non-cleaved cell lymphoma, and small lymphocytic lymphoma.

The term “leukemia” as used herein refers to any of the various acute or chronic neoplastic disease of the bone marrow characterized by proliferation or abnormal increases in the number of white blood cells. The many types of leukemia are typically classified according to the type of white blood cell involved. Examples of this group of leukemia includes, but are not limited to, myeloid leukemia [acute and chronic], acute lymphoblastic leukemia, small lymphocytic leukemia and chronic lymphocytic leukemia.

The term “treatment of B-cell lymphoma” or “treatment of leukemia” as referred to in this description means that an anti-cancer agent is administered to a patient diagnosed with a B-cell lymphoma or leukemia so as to inhibit the growth of the malignant cells in the patient.

The term “patient” or “subject” as referred to in this description means the recipient in need of medical intervention or treatment. Mammalian and non-mammalian patients or subjects are included.

The term “predictive biomarker” as referred to in this description means a gene marker whose expression is correlated with a response to a given therapeutic agent or class of therapeutic agents.

“Marker-derived polynucleotides” means the RNA transcribed from a marker gene, any cDNA or cRNA produced there from, and any nucleic acid derived there from, such as synthetic nucleic acid having a sequence derived from the gene corresponding to the marker gene.

The terms “control,” “control level,” “reference level,” or “pre-determined reference level” means a separate baseline level measured in a comparable control cell, which may or may not be disease free. It may be from the same individual or from another individual who is normal or does not present with the same disease from which the disease or test sample is obtained. Thus, “reference value” can be an absolute value, a range of values, an average value, a median value, a mean value, or a value as compared to a particular control or baseline value. A reference value can be based on an individual sample value, such as, a value obtained from a sample from an individual with a B-cell lymphoma or leukemia, but at an earlier point in time or prior to treatment, or a value obtained from a sample from a patient diagnosed with a B-cell lymphoma or leukemia other than the individual being tested, or a “normal” individual, that is an individual not diagnosed with a B-cell lymphoma or leukemia. The reference value can be based on a number of samples, such as from multiple patients diagnosed with a B-cell lymphoma or leukemia, or normal individuals, or based on a pool of samples including or excluding the sample to be tested.

The term “CD86” as referred to in this description means the gene that encodes the type I membrane protein, which is a member of the immunoglobulin superfamily. This protein is expressed by antigen-presenting cells and is the ligand for two proteins at the surface of T cells, CD28 antigen and cytotoxic T-lymphocyte-associated protein 4. Alternative splicing results in several transcript variants (variants 1-5), the longest of which is variant 1 (NCBI Ref No. NM_175862 (SEQ ID NO:1) and NP_787058 (SEQ ID NO:2)). The mRNA and protein sequences for variants 2-5, as well as for variant 1, can be found under NCBI Transcript Reference Numbers, NM_006889 (SEQ ID NO:3)/NP_008820 (SEQ ID NO:4), NM_176892 (SEQ ID NO:5)/NP_795711 (SEQ ID NO:6), NM_001206924 (SEQ ID NO:7)/NP_001193853 (SEQ ID NO:8), and NM_001206925 (SEQ ID NO:9)/NP_001193854 (SEQ ID NO:10), respectively, which are incorporated herein by reference in their entirety.

The term “high expression of CD86” or “elevated CD86 expression” as referred to in this description means having higher CD86 DNA, mRNA, or protein expression, or an increase in the number of copies of the CD86 gene in a cell from a patient diagnosed with B-cell lymphoma or leukemia, or cell obtained from a B-cell lymphoma or leukemia cell line, as compared to a cell from a patient not diagnosed with B-cell lymphoma or leukemia, or a cell obtained from a cell line not characterized by B-cell lymphoma or leukemia, or a control cell; or from a cell of a patient diagnosed with B-cell lymphoma or leukemia but does not respond to treatment with a SYK inhibitor.

As used herein, the terms “measuring expression levels,” “measuring gene expression level,” or “obtaining an expression level” and the like, includes methods that quantify target gene expression level exemplified by a transcript of a gene, including mRNA, microRNA (miRNA) or a protein encoded by a gene, as well as methods that determine whether a gene of interest is expressed at all. Thus, an assay which provides a “yes” or “no” result without necessarily providing quantification of an amount of expression is an assay that “measures expression” as that term is used herein. Alternatively, the term may include quantifying expression level of the target gene expressed in a quantitative value, for example, a fold-change in expression, up or down, relative to a control gene or relative to the same gene in another sample, or a log ratio of expression, or any visual representation thereof, such as, for example, a “heatmap” where a color intensity is representative of the amount of gene expression detected. Exemplary methods for detecting the level of expression of a gene include, but are not limited to, Northern blotting, dot or slot blots, reporter gene matrix (see, for example, U.S. Pat. No. 5,569,588), nuclease protection, RT-PCR, microarray profiling, differential display, SAGE (Velculescu et al., (1995), Science 270:484-87), Digital Gene Expression System (see WO2007076128; WO2007076129), multiplex mRNA assay (Tian et al., (2004), Nucleic Acids Res. 32:e126), PMAGE (Kim et al., (2007), Science 316:1481-84), cDNA-mediated annealing, selection, extension and ligation assay (DASL, Bibikova, et al., (2004), AJP 165:1799-807), multiplex branched DNA assay (Flagella et al., (2006), Anal. Biochem. 352:50-60), 2D gel electrophoresis, SELDI-TOF, ICAT, enzyme assay, antibody assay, and the like.

### SYK Inhibitors

In an embodiment of the invention, the SYK inhibitor administered is any of the compounds exemplified in WO 2012/154519, which is incorporated by reference herein in its entirety. SYK-1 utilized to identify and validate the predictive biomarker claimed herein is one of the compounds described in this application. In another embodiment, the SYK inhibitor is selected from the group consisting of trans-4-{[5-(3-{[4-(difluoromethyl)pyrimidin-2-yl]amino}-5-methylphenyl)-1,3-thiazol-2-yl]-1-hydroxyethyl}cyclohexanecarboxylic acid; trans-4-{1-hydroxy-1-[5-(3-methyl-5-{[4-(trifluoromethyl)pyrimidin-2-yl]amino}phenyl)-1,3-thiazol-2-yl]ethyl}cyclohexanecarboxylic acid; 4-[1-hydroxy-1-(5-{3-methyl-5-[(4-methylpyrimidin-2-yl)amino]phenyl}-1,3-thiazol-2-yl)ethyl]-2-methylcyclohexanecarboxylic acid; 4-[1-hydroxy-1-(5-{3-[(4-methoxypyrimidin-2-yl)amino]-5-methylphenyl}-1,3-thiazol-2-yl)ethyl]-2-methylcyclohexanecarboxylic acid; and 4-{1-[5-(3-{[4-(difluoromethyl)pyrimidin-2-yl]amino}-5-methylphenyl)-1,3-thiazol-2-yl]-1-hydroxyethyl}-2-methylcyclohexanecarboxylic acid, or a stereoisomer, or a pharmaceutically acceptable salt thereof.

Compounds of WO 2012/154519 have been described as having SYK activity (rhSyk activity (IC₅₀)) based on a homogeneous time-resolved fluorescence (HTRF) assay, incorporated herein as Example 2 using a recombinant human SYK fusion protein. SYK activity for the compounds therein was classified as follows:

+++ 100 nM or less

++ between 100 and 1000 nM

+ between 1 and 10 μM

SYK-1 is one of the compounds described in WO 2012/154519 as having +++ SYK activity. IC₅₀ values for representative compounds of WO 2012/154519 are as follows:

| Example/Compound Number | rhSyk (nM) |
|---|---|
| Example 1 (faster eluting isomer) | <0.5 |
| Example 1 (slower eluting enantiomer) | 2 |
| 1-3 | 1 |
| 1-11 | 3 |
| 1-18 | <0.5 |
| 2-1 | <0.5 |
| 2-3 | 1258 |
| 2-16 | 93 |
| 2-52 | 1 |
| 2-53 | 13 |
| 2-57 | 123 |
| 2-60 | 5 |
| 2-73 | 1460 |
| 2-74 | 1429 |
| 2-77 | <0.5 |
| 2-79 | 2 |
| 2-101 | 2 |
| 2-109 | 15 |
| 2-110 | 1 |
| 2-112 | 3 |
| 2-114 | 1 |
| 3-2 | 2 |
| 4-6 | 2 |
| Example 5, Step 3 (trans isomer) | 318 |
| 6-3 | 5 |
| Example 7 (trans isomer) | 35 |
| Example 13, step 2 | 301 |
| Example 14 | 850 |
| Example 15 | <0.5 |
| Example 16 | 1 |
| Example 17 | 1 |
| Example 18 | <0.5 |
| Example 22 | 197 |

While the methods of the invention herein have been exemplified using a SYK inhibitor compound described in WO 2012/154519, those of ordinary skill in the art would recognize and appreciate that any SYK inhibitor compound having the requisite SYK activity could be used to treat a patient diagnosed with a B-cell lymphoma or leukemia. In one embodiment, patients who are identified as likely to respond to a SYK inhibitor, that is, who are characterized by high or elevated levels of CD86 are to be treated with a SYK inhibitor compound having +++SYK activity (IC₅₀ 100 nM or less).

In another embodiment of the invention, the SYK inhibitor is any of the compounds exemplified in U.S. Pat. No. 8,551,984, which is incorporated by reference herein in its entirety. In one embodiment, the SYK inhibitor is selected from the group consisting of 5-hydroxy-5-[5-(3-methyl-5-{[4-(trifluoromethyl)pyrimidin-2-yl]amino}phenyl)-1,3-thiazol-2-yl]azepan-2-one, (1S,4R)-4-hydroxy-2,2-dimethyl-4-[5-(3-methyl-5-{[4-(trifluoromethyl)pyrimidin-2-yl]amino}phenyl)-1,3-thiazol-2-yl]-N-[3-(2-oxopyrrolidin-1-yl)propyl]cyclohexanecarboxamide, cis-4-[(hydroxyacetyl)amino]-1-[5-(3-methyl-5-{[4-(trifluoromethyl)pyrimidin-2-yl]amino}phenyl)-1,3-thiazol-2-yl]cyclohexanecarboxamide, (1S,4R)-4-hydroxy-2,2-dimethyl-4-[5-(3-methyl-5-{[4-(trifluoromethyl)pyrimidin-2-yl]amino}phenyl)-1,3-thiazol-2-yl]cyclohexanecarboxylic acid, (1S,4R)-4-{5-[3-({4-[(1S)-1-fluoroethyl]pyrimidin-2-yl}amino)-5-methylphenyl]-1,3-thiazol-2-yl}-4-hydroxy-2,2-dimethylcyclohexanecarboxylic acid, (1S,4R)-4-{5-[3-({4-[(1R)-1-fluoroethyl]pyrimidin-2-yl}amino)-5-methylphenyl]-1,3-thiazol-2-yl}-4-hydroxy-2,2-dimethylcyclohexanecarboxylic acid, and (1S,4R)-4-hydroxy-2,2-dimethyl-4-{5-[3-methyl-5-(4-methyl-pyrimidin-2-ylamino)-phenyl]-1,3-thiazol-2-yl}-cyclohexanecarboxylic acid or a stereoisomer, or a pharmaceutically acceptable salt thereof.

In another embodiment of the invention, the SYK inhibitor is any of the compounds exemplified in WO2013/192125, which is incorporated by reference herein in its entirety. In one embodiment, the SYK inhibitor is selected from the group consisting of 3-[4-(3-{[4-(difluoromethyl)pyrimidin-2-yl]amino}-5-methylphenyl)-1H-pyrazol-1-yl]cyclohexane-1,2-diol; 5-{[4-(3-{[4-(difluoromethyl)-5-fluoropyrimidin-2-yl]amino}-5-methylphenyl)-1H-pyrazol-1-yl]methyl}-4-methyl-1,3-oxazolidin-2-one; 5-{[4-(3-{[4-(difluoromethyl)pyrimidin-2-yl]amino}-5-methylphenyl)-1H-pyrazol-1-yl]methyl}pyridin-2(1H)-one; and 3-(4-{3-methyl-5-[(4-methylpyrimidin-2-yl)amino]phenyl}-1H-pyrazol-1-yl)cyclohexane-1,2-diol, or a stereoisomer, or a pharmaceutically acceptable salt thereof.

The SYK inhibitor administered in the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention. In addition, the compounds disclosed herein may exist as tautomers and both tautomeric forms are intended to be encompassed by the scope of the invention, even though only one tautomeric structure is depicted.

In the SYK inhibitor administered in the present invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds disclosed herein. For example, different isotopic forms of hydrogen (H) include protium (1H) and deuterium (2H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds disclosed herein can be prepared without undue experimentation by conventional techniques well known to those skilled in the art using appropriate isotopically-enriched reagents and/or intermediates.

The SYK inhibitor administered in the instant invention may also exist as various crystals, amorphous substances, pharmaceutically acceptable salts, hydrates and solvates. Further, the SYK inhibitors administered in the instant invention may be provided as prodrugs. In general, such prodrugs are functional derivatives of the SYK inhibitors administered in the instant invention that can be readily converted into compounds that are needed by living bodies. Accordingly, in the method of treatment of various cancers in the invention, the term “administration” includes not only the administration of a specific compound but also the administration of a compound which, after administered to patients, can be converted into the specific compound in the living bodies. Conventional methods for selection and production of suitable prodrug derivatives are described, for example, in “Design of Prodrugs”, ed. H. Bundgaard, Elsevier, 1985, which is referred to herein and is entirely incorporated herein as a part of the present description. Metabolites of the compound may include active compounds that are produced by putting the compound in a biological environment, and are within the scope of the compound administered in the invention.

### Methods of Measuring a Biomarker

In one embodiment, the invention is a predictive biomarker, CD86, whose expression is sensitive to SYK inhibition by a SYK inhibitor. The expression levels of the predictive biomarker in a sample may be determined by any means known in the art. The expression level may be determined by isolating and determining the level (i.e., amount) of nucleic acid transcribed from the biomarker. Alternatively, or additionally, the level of specific proteins encoded by the biomarker may be determined.

The level of expression of a biomarker can be accomplished by determining the amount of mRNA, or other polynucleotides derived from the biomarker, present in a sample. Any method for determining RNA levels can be used. For example, RNA is isolated from a sample and separated on an agarose gel. The separated RNA is then transferred to a solid support, such as a filter. Nucleic acid probes representing one or more markers are then hybridized to the filter by northern hybridization, and the amount of marker-derived RNA is determined. Such determination can be visual, or machine-aided, for example, by use of a densitometer. Another method of determining RNA levels is by use of a dot-blot or a slot-blot. In this method, RNA, or nucleic acid derived therefrom, from a sample is labeled. The RNA or nucleic acid derived therefrom is then hybridized to a filter containing oligonucleotides derived from one or more marker genes, wherein the oligonucleotides are placed upon the filter at discrete, easily-identifiable locations. Hybridization, or lack thereof, of the labeled RNA to the filter-bound oligonucleotides is determined visually or by densitometer. Polynucleotides can be labeled using a radiolabel or a fluorescent (i.e., visible) label.

The expression of a biomarker gene in a number of tissue specimens may be characterized using a “tissue array” (Kononen et al., Nat. Med, 1998, 4(7):844-847). In a tissue array, multiple tissue samples may be assessed on the same microarray. The tissue array allow in situ detection of RNA and protein levels; consecutive sections allow the analysis of multiple samples simultaneously.

To determine the (high or low) expression level of CD86 in the practice of the present invention, any method known in the art may be utilized. In one embodiment of the invention, expression based on detection of RNA which hybridizes to the gene identified and disclosed herein is used. This is readily performed by any RNA detection or amplification methods known or recognized as equivalent in the art such as, but not limited to, reverse transcription-PCR, and methods to detect the presence, or absence, of RNA stabilizing or destabilizing sequences. These examples are not intended to be limiting, as other methods of determining RNA abundance are known in the art.

Alternatively, expression based on detection of DNA status may be used. Detection of the DNA of an identified gene as may be used for genes that have increased expression in correlation with a particular outcome. This may be readily performed by PCR based methods known in the art, including, but not limited to, Q-PCR. Conversely, detection of the DNA of an identified gene as amplified may be used for genes that have increased expression in correlation with a particular treatment outcome. This may be readily performed by PCR based, fluorescent in situ hybridization (FISH) and chromosome in situ hybridization (CISH), the Roche AmpliChip, and methods known in the art.

### B. Microarrays

In some embodiments, polynucleotide microarrays may be used to measure expression or gene amplification so that the expression or amplification status of each biomarker is assessed simultaneously. When used in a specific embodiment, the invention provides polynucleotide arrays in which the biomarkers identified for a particular subject subset comprise at least 50%, 60%, 70%, 80%, 85%, 90%, 95% or 98% of the probes on said array. In another specific embodiment, the microarray comprises a plurality of probes, wherein said plurality of probes comprise probes complementary and hybridizable to at least CD86, and optionally one or more of the SYK inhibitor exposure/prediction-informative biomarkers identified for a particular patient subset. Microarrays of the invention, of course, may comprise probes complementary to and which are capable of hybridizing to CD86 and optionally one or more SYK inhibitor prediction/evaluation-informative biomarkers for a plurality of the subject subsets, or for each subject subset, identified for a particular condition. In furtherance thereof, a microarray of the invention comprises a plurality of probes complementary to and which hybridize to CD86, and optionally one or more SYK inhibitor prediction/evaluation-informative biomarkers identified for each subject subset identified for the condition of interest, and wherein said probes, in total, are at least 50% of the probes on said microarray.

In yet another specific embodiment, the microarray is a commercially-available cDNA microarray that comprises CD86, and one or more predictive biomarkers identified by the methods described herein. A commercially-available cDNA microarray comprises CD86 and one or more biomarkers identified by the methods described herein as being informative for a patient subset for a particular condition. However, such a microarray may comprise at least 1, 2, 3, 4 or 5 of such markers, up to the maximum number of markers identified.

Any of the microarrays described herein may be provided in a sealed container in a kit.

### C. Polynucleotides Used to Measure the Products of the Predictive Biomarker

Polynucleotides capable of specifically or selectively binding to the mRNA transcripts encoding the polypeptide predictive biomarker, CD86, of the invention are also contemplated. For example: oligonucleotides, cDNA, DNA, RNA, PCR products, synthetic DNA, synthetic RNA, or other combinations of naturally occurring or modified nucleotides which specifically and/or selectively hybridize to one or more of the RNA products of the predictive biomarker of the invention are useful in accordance with the invention.

In a preferred embodiment, the oligonucleotides, cDNA, DNA, RNA, PCR products, synthetic DNA, synthetic RNA, or other combinations of naturally occurring or modified nucleotides oligonucleotides which both specifically and selectively hybridize to one or more of the RNA products of the predictive biomarker of the invention are used.

### D. Techniques to Measure the RNA Products of a Biomarker

### 1. Real-Time PCR

In practice, a gene expression-based expression assay based on a small number of genes, i.e., about 1 to 3000 genes can be performed with relatively little effort using existing quantitative real-time PCR technology familiar to clinical laboratories. Quantitative real-time PCR measures PCR product accumulation through a dual-labeled fluorigenic probe. A variety of normalization methods may be used, such as an internal competitor for each target sequence, a normalization gene contained within the sample, or a housekeeping gene. Sufficient RNA for real time PCR can be isolated from low milligram quantities from a subject. Quantitative thermal cyclers may now be used with microfluidics cards preloaded with reagents making routine clinical use of multigene expression-based assays a realistic goal.

The predictive biomarker assayed according to the present invention, are typically in the form of total RNA or mRNA or reverse transcribed total RNA or mRNA. General methods for total and mRNA extraction are well known in the art and are disclosed in standard textbooks of molecular biology, including Ausubel et al., Current Protocols of Molecular Biology, John Wiley and Sons (1997). RNA isolation can also be performed using purification kit, buffer set and protease from commercial manufacturers, such as Qiagen (Valencia, Calif.) and Ambion (Austin, Tex.), according to the manufacturer's instructions.

TAQman quantitative real-time PCR can be performed using commercially available PCR reagents (Applied Biosystems, Foster City, Calif.) and equipment, such as ABI Prism 7900HT Sequence Detection System (Applied Biosystems) according the manufacturer's instructions. The system consists of a thermocycler, laser, charge-coupled device (CCD), camera, and computer. The system amplifies samples in a 96-well or 384-well format on a thermocycler. During amplification, laser-induced fluorescent signal is collected in real-time through fiber-optic cables for all 96 wells, and detected at the CCD. The system includes software for running the instrument and for analyzing the data.

Based upon the predictive biomarker identified in the present invention, a real-time PCR TAQman assay can be used to make gene expression measurements and perform the classification methods described herein. As is apparent to a person of skill in the art, a wide variety of oligonucleotide primers and probes that are complementary to or hybridize to the predictive biomarker of the invention may be selected based upon the predictive biomarker transcript sequence.

### 2. Array Hybridization

The polynucleotide used to measure the RNA products of the invention can be used as nucleic acid members stably associated with a support to comprise an array according to one aspect of the invention. The length of a nucleic acid member can range from 8 to 1000 nucleotides in length and are chosen so as to be specific for the RNA products of the predictive biomarker of the invention. In one embodiment, these members are selective for the RNA products of the invention. The nucleic acid members may be single or double stranded, and/or may be oligonucleotides or PCR fragments amplified from cDNA. Preferably oligonucleotides are approximately 20-30 nucleotides in length. ESTs are preferably 100 to 600 nucleotides in length. It will be understood to a person skilled in the art that one can utilize portions of the expressed regions of the predictive biomarker of the invention as a probe on the array. More particularly oligonucleotides complementary to the genes of the invention and cDNA or ESTs derived from the genes of the invention are useful. For oligonucleotide based arrays, the selection of oligonucleotides corresponding to the gene of interest which are useful as probes is well understood in the art. More particularly it is important to choose regions which will permit hybridization to the target nucleic acids. Factors such as the Tm of the oligonucleotide, the percent GC content, the degree of secondary structure and the length of nucleic acid are important factors. See for example U.S. Pat. No. 6,551,784.

### 3. Construction of a Nucleic Acid Array

In the proposed methods, an array of nucleic acid members stably associated with the surface of a substantially support is contacted with a sample comprising target nucleic acids under hybridization conditions sufficient to produce a hybridization pattern of complementary nucleic acid members/target complexes in which one or more complementary nucleic acid members at unique positions on the array specifically hybridize to target nucleic acids. The identity of target nucleic acids which hybridize can be determined with reference to location of nucleic acid members on the array.

The nucleic acid members may be produced using established techniques such as polymerase chain reaction (PCR) and reverse transcription (RT). These methods are similar to those currently known in the art (see, for example, PCR Strategies, Michael A. Innis (Editor), et al., 1995 and PCR: Introduction to Biotechniques Series, C. R. Newton, A. Graham, 1997). Amplified nucleic acids are purified by methods well known in the art (e.g., column purification or alcohol precipitation). A nucleic acid is considered pure when it has been isolated so as to be substantially free of primers and incomplete products produced during the synthesis of the desired nucleic acid. Preferably, a purified nucleic acid will also be substantially free of contaminants which may hinder or otherwise mask the specific binding activity of the molecule.

An array, according to one aspect of the invention, comprises a plurality of nucleic acids attached to one surface of a support at a density exceeding 20 different nucleic acids/cm², wherein each of the nucleic acids is attached to the surface of the support in a non-identical pre-selected region (e.g. a microarray). Each associated sample on the array comprises a nucleic acid composition, of known identity, usually of known sequence, as described in greater detail below. Any conceivable substrate may be employed in the invention.

In one embodiment, the nucleic acid attached to the surface of the support is DNA. In one embodiment, the nucleic acid attached to the surface of the support is cDNA or RNA. In another embodiment, the nucleic acid attached to the surface of the support is cDNA synthesized by polymerase chain reaction (PCR). Usually, a nucleic acid member in the array, according to the invention, is at least 10, 25, 50, 60 nucleotides in length. In one embodiment, a nucleic acid member is at least 150 nucleotides in length. Preferably, a nucleic acid member is less than 1000 nucleotides in length. More preferably, a nucleic acid member is less than 500 nucleotides in length.

In the arrays of the invention, the nucleic acid compositions are stably associated with the surface of a support, where the support may be a flexible or rigid support. By “stably associated” is meant that each nucleic acid member maintains a unique position relative to the support under hybridization and washing conditions. As such, the samples are non-covalently or covalently stably associated with the support surface. Examples of non-covalent association include non-specific adsorption, binding based on electrostatic interactions (e.g., ion pair interactions), hydrophobic interactions, hydrogen bonding interactions, specific binding through a specific binding pair member covalently attached to the support surface, and the like. Examples of covalent binding include covalent bonds formed between the nucleic acids and a functional group present on the surface of the rigid support (e.g., —OH), where the functional group may be naturally occurring or present as a member of an introduced linking group, as described in greater detail below.

The amount of nucleic acid present in each composition will be sufficient to provide for adequate hybridization and detection of target nucleic acid sequences during the assay in which the array is employed. Generally, the amount of each nucleic acid member stably associated with the support of the array is at least about 0.001 ng, preferably at least about 0.02 ng and more preferably at least about 0.05 ng, where the amount may be as high as 1000 ng or higher, but will usually not exceed about 20 ng. Where the nucleic acid member is “spotted” onto the support in a spot comprising an overall circular dimension, the diameter of the “spot” will generally range from about 10 to 5,000 μm, usually from about 20 to 2,000 μm and more usually from about 100 to 200 μm.

Control nucleic acid members may be present on the array including nucleic acid members comprising oligonucleotides or nucleic acids corresponding to genomic DNA, housekeeping genes, vector sequences, plant nucleic acid sequence, negative and positive control genes, and the like. Control nucleic acid members are calibrating or control genes whose function is not to tell whether a particular “key” gene of interest is expressed, but rather to provide other useful information, such as background or basal level of expression.

Other control nucleic acids are spotted on the array and used as target expression control nucleic acids and mismatch control nucleotides to monitor non-specific binding or cross-hybridization to a nucleic acid in the sample other than the target to which the probe is directed. Mismatch probes thus indicate whether a hybridization is specific or not. For example, if the target is present, the perfectly matched probes should be consistently brighter than the mismatched probes. In addition, if all control mismatches are present, the mismatch probes are used to detect a mutation.

Numerous methods may be used for attachment of the nucleic acid members of the invention to the substrate (a process referred to as “spotting”). For example, nucleic acids are attached using the techniques of, for example U.S. Pat. No. 5,807,522, which is incorporated herein by reference for teaching methods of polymer attachment. Alternatively, spotting may be carried out using contact printing technology as is known in the art.

The measuring of the expression of the RNA product of the invention can be done by using those polynucleotides which are specific and/or selective for the RNA products of the invention to quantitate the expression of the RNA product. In a specific embodiment of the invention, the polynucleotides which are specific and/or selective for the RNA products are probes or primers. In one embodiment, these polynucleotides are in the form of nucleic acid probes which can be spotted onto an array to measure RNA from the sample of an individual to be measured. In another embodiment, commercial arrays can be used to measure the expression of the RNA product. In yet another embodiment, the polynucleotides which are specific and/or selective for the RNA products of the invention are used in the form of probes and primers in techniques such as quantitative real-time RT PCR, using for example SYBR®Green, or using TaqMan® or Molecular Beacon techniques, where the polynucleotides used are used in the form of a forward primer, a reverse primer, a TaqMan labeled probe or a Molecular Beacon labeled probe.

In embodiments where only one or a two genes are to be analyzed, the nucleic acid derived from the sample cell(s) may be preferentially amplified by use of appropriate primers such that only the genes to be analyzed are amplified to reduce background signals from other genes expressed in the breast cell. Alternatively, and where multiple genes are to be analyzed or where very few cells (or one cell) is used, the nucleic acid from the sample may be globally amplified before hybridization to the immobilized polynucleotides. Of course RNA, or the cDNA counterpart thereof may be directly labeled and used, without amplification, by methods known in the art.

4. Use of a microarray

A “microarray” is a linear or two-dimensional array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, preferably at least about 50/cm², more preferably at least about 100/cm², even more preferably at least about 500/cm², but preferably below about 1,000/cm². Preferably, the arrays contain less than about 500, about 1000, about 1500, about 2000, about 2500, or about 3000 immobilized polynucleotides in total. As used herein, a DNA microarray is an array of oligonucleotides or polynucleotides placed on a chip or other surfaces used to hybridize to amplified or cloned polynucleotides from a sample. Since the position of each particular group of primers in the array is known, the identities of a sample polynucleotides can be determined based on their binding to a particular position in the microarray.

Determining gene expression levels may be accomplished utilizing microarrays. Generally, the following steps may be involved: (a) obtaining an mRNA sample from a subject and preparing labeled nucleic acids therefrom (the “target nucleic acids” or “targets”); (b) contacting the target nucleic acids with an array under conditions sufficient for the target nucleic acids to bind to the corresponding probes on the array, for example, by hybridization or specific binding; (c) optional removal of unbound targets from the array; (d) detecting the bound targets, and (e) analyzing the results, for example, using computer based analysis methods. As used herein, “nucleic acid probes” or “probes” are nucleic acids attached to the array, whereas “target nucleic acids” are nucleic acids that are hybridized to the array.

A nucleic acid specimen may be obtained from a subject to be tested using either “invasive” or “non-invasive” sampling means. A sampling means is said to be “invasive” if it involves the collection of nucleic acids from within the skin or organs of an animal (including murine, human, ovine, equine, bovine, porcine, canine, or feline animal). Examples of an invasive sampling means include, blood collection, semen collection, needle biopsy, pleural aspiration, umbilical cord biopsy. Examples of such methods are discussed by Kim, et al., J. Virol., 1992, 66:3879-3882, Biswas, et al., Ann. NY Acad. Sci., 1990, 590:582-583, and Biswas, et al., J. Clin. Microbiol., 1991, 29:2228-2233.

In contrast, a “non-invasive” sampling means is one in which the nucleic acid molecules are recovered from an internal or external surface of the animal. Examples of a “non-invasive” sampling means include, “swabbing,” collection of tears, saliva, urine, fecal material, or the like.

In one embodiment of the present invention, one or more cells, i.e. a sample, from a subject to be tested are obtained and RNA is isolated from the cells. It is also possible to obtain a cell sample from a subject, and then to enrich the sample for a desired cell type. For example, cells may be isolated from other cells using a variety of techniques, such as isolation with an antibody binding to an epitope on the cell surface of the desired cell type. Where the desired cells are in a solid tissue, particular cells may be dissected, for example, by micro-dissection or by laser capture micro-dissection (LCM) (see, e.g., Bonner, et al., Science, 1997, 278:1481, Emmert-Buck, et al., Science, 1996, 274:998, Fend, et al., Am. J. Path., 1999, 154:61, and Murakami, et al., Kidney Hit., 2000, 58:1346.

RNA may be extracted from tissue or cell samples by a variety of methods, for example, guanidium thiocyanate lysis followed by CsCl centrifugation (Chirgwin, et al., Biochemistry, 1979, 18:5294-5299). RNA from single cells may be obtained as described in methods for preparing cDNA libraries from single cells (see, e.g., Dulac, Curr. Top. Dev. Biol., 1998, 36:245, and Jena, et al., J. Immunol. Methods, 1996, 190:199).

The RNA sample can be further enriched for a particular species. In one embodiment, for example, poly(A)+RNA may be isolated from an RNA sample. In another embodiment, the RNA population may be enriched for sequences of interest by primer-specific cDNA synthesis, or multiple rounds of linear amplification based on cDNA synthesis and template-directed in vitro transcription (see, e.g., Wang, et al., Proc. Natl. Acad. Sci. USA, 1989, 86:9717; Dulac, et al., supra; Jena, et al., supra). In addition, the population of RNA, enriched or not in particular species or sequences, may be further amplified by a variety of amplification methods including, PCR, ligase chain reaction (LCR) (see, e.g., Wu and Wallace, Genomics, 1989, 4:560; Landegren, et al., Science, 1988, 241:1077), self-sustained sequence replication (SSR) (see, e.g., Guatelli, et al., Proc. Natl. Acad. Sci. USA, 1990, 87:1874), nucleic acid based sequence amplification (NASBA) and transcription amplification (see, e.g., Kwoh, et al., Proc. Natl. Acad. Sci. USA, 1989, 86:1173). Methods for PCR technology are well known in the art (see, e.g., PCR Technology: Principles and Applications for DNA Amplification, ed. H. A. Erlich, Freeman Press, N.Y., N.Y., 1992; PCR Protocols: A Guide to Methods and Applications, eds. Innis, et al., Academic Press, San Diego, Calif., 1990; Mattila, et al., Nucleic Acids Res., 1991, 19:4967; Eckert, et al., PCR Methods and Applications, 1991, 1:17; PCR, eds. McPherson et al., IRL Press, Oxford; and U.S. Pat. No. 4,683,202). Methods of amplification are described, for example, by Ohyama, et al., BioTechniques, 2000, 29:530; Luo, et al., Nat. Med., 1999, 5:117; Hegde, et al., BioTechniques, 2000, 29:548; Kacharmina, et al., Meth. Enzymol., 1999, 303:3; Livesey, et al., Curr. Biol., 2000, 10:301; Spirin, et al., Invest. Ophtalmol. Vis. Sci., 1999, 40:3108; and Sakai, et al., Anal. Biochem., 2000, 287:32. RNA amplification and cDNA synthesis may also be conducted in cells in situ (see, e.g., Eberwine, et al., Proc. Natl. Acad. Sci. USA, 1992, 89:3010).

In yet another embodiment of the invention, all or part of a disclosed marker sequence may be amplified and detected by methods such as the polymerase chain reaction (PCR) and variations thereof, such as, but not limited to, quantitative PCR (Q-PCR), reverse transcription PCR (RT-PCR), and real-time PCR, optionally real-time RT-PCR. Such methods would utilize one or two primers that are complementary to portions of a disclosed sequence, where the primers are used to prime nucleic acid synthesis.

The newly synthesized nucleic acids are optionally labeled and may be detected directly or by hybridization to a polynucleotide of the invention.

The nucleic acid molecules may be labeled to permit detection of hybridization of the nucleic acid molecules to a microarray. That is, the probe may comprise a member of a signal producing system and thus, is detectable, either directly or through combined action with one or more additional members of a signal producing system. For example, the nucleic acids may be labeled with a fluorescently labeled dNTP (see, e.g., Kricka, Nonisotopic DNA Probe Techniques, Academic Press San Diego, Calif., 1992), biotinylated dNTPs or rNTP followed by addition of labeled streptavidin, chemiluminescent labels, or isotopes. Another example of labels includes “molecular beacons” as described in Tyagi and Kramer, Nature Biotech., 1996, 14:303. The newly synthesized nucleic acids may be contacted with polynucleotides (containing sequences) of the invention under conditions which allow for their hybridization. Hybridization may be also determined, for example, by plasmon resonance (see, e.g., Thiel, et al., Anal. Chem., 1997, 69:4948).

In one embodiment, a plurality, for example, two sets of target nucleic acids are labeled and used in one hybridization reaction (“multiplex” analysis). One set of nucleic acids may correspond to RNA from one cell and another set of nucleic acids may correspond to RNA from another cell. The plurality of sets of nucleic acids may be labeled with different labels, such as different fluorescent labels (e.g., fluorescein and rhodamine), which have distinct emission spectra so that they can be distinguished. The sets may then be mixed and hybridized simultaneously to one microarray (see, e.g., Shena, et al., Science, 1995, 270:467-470).

A number of different microarray configurations and methods for their production are known to those of skill in the art and are disclosed in U.S. Pat. Nos. 5,242,974; 5,384,261; 5,405,783; 5,412,087; 5,424,186; 5,429, 807; 5,436,327; 5,445,934; 5,556,752; 5,405,783; 5,412,087; 5,424,186; 5, 429,807; 5,436,327; 5,472,672; 5,527,681; 5,529,756; 5,545,531; 5,554,501; 5,561,071; 5,571,639; 5,593,839; 5,624,711; 5, 700,637; 5,744,305; 5,770,456; 5,770,722; 5,837,832; 5,856,101; 5,874,219; 5,885,837; 5,919,523; 6,022,963; 6,077,674; and 6,156,501; Shena, et al., Tibtech 16:301, 1998; Duggan, et al., Nat. Genet. 21:10, 1999; Bowtell, et al., Nat. Genet. 21:25, 1999; Lipshutz, et al., 21 Nature Genet. 20-24, 1999; Blanchard, et al., 11 Biosensors and Bioelectronics, 687-90, 1996; Maskos, et al., 21 Nucleic Acids Res. 4663-69, 1993; Hughes, et al., Nat. Biotechol. (2001) 19:342. Patents describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,848,659; and 5,874,219.

In one embodiment, an array of oligonucleotides may be synthesized on a solid support. Exemplary solid supports include glass, plastics, polymers, metals, metalloids, ceramics, organics, etc. Using chip masking technologies and photoprotective chemistry, it is possible to generate ordered arrays of nucleic acid probes. These arrays, which are known, for example, as “DNA chips” or very large scale immobilized polymer arrays (“VLSIPS®” arrays), may include millions of defined probe regions on a substrate having an area of about 1 cm² to several cm², thereby incorporating from a few to millions of probes (see, e.g., U.S. Pat. No. 5,631,734).

To compare expression levels, labeled nucleic acids may be contacted with the array under conditions sufficient for binding between the target nucleic acid and the probe on the array. In one embodiment, the hybridization conditions may be selected to provide for the desired level of hybridization specificity; that is, conditions sufficient for hybridization to occur between the labeled nucleic acids and probes on the microarray.

Hybridization may be carried out in conditions permitting essentially specific hybridization. The length and GC content of the nucleic acid will determine the thermal melting point and thus, the hybridization conditions necessary for obtaining specific hybridization of the probe to the target nucleic acid. These factors are well known to a person of skill in the art, and may also be tested in assays. An extensive guide to nucleic acid hybridization may be found in Tijssen, et al., Laboratory Techniques in Biochemistry and Molecular Biology, Vol. 24: Hybridization with Nucleic Acid Probes, P. Tijssen, ed. Elsevier, N. Y., 1993.

The methods described above will result in the production of hybridization patterns of labeled target nucleic acids on the array surface. The resultant hybridization patterns of labeled nucleic acids may be visualized or detected in a variety of ways, with the particular manner of detection selected based on the particular label of the target nucleic acid. Representative detection means include scintillation counting, autoradiography, fluorescence measurement, calorimetric measurement, light emission measurement, light scattering, and the like.

One such method of detection utilizes an array scanner that is commercially available (Affymetrix, Santa Clara, Calif.), for example, the 417® Arrayer, the 418® Array Scanner, or the Agilent GeneArray® Scanner. This scanner is controlled from a system computer with an interface and easy-to-use software tools. The output may be directly imported into or directly read by a variety of software applications. Exemplary scanning devices are described in, for example, U.S. Pat. Nos. 5,143,854 and 5,424,186.

### Dosing and Routes of Administration

With regard to the SYK inhibitors used in the invention, various preparation forms can be selected, and examples thereof include oral preparations such as tablets, capsules, powders, granules or liquids, or sterilized liquid parenteral preparations such as solutions or suspensions, suppositories, ointments and the like. The SYK inhibitors are available as pharmaceutically acceptable salts. The SYK inhibitors used in the invention are prepared with pharmaceutically acceptable carriers or diluents.

The term “pharmaceutically acceptable salt” as referred to in this description means ordinary, pharmaceutically acceptable salt. For example, when the compound has a hydroxyl group, or an acidic group such as a carboxyl group and a tetrazolyl group, then it may form a base-addition salt at the hydroxyl group or the acidic group; or when the compound has an amino group or a basic heterocyclic group, then it may form an acid-addition salt at the amino group or the basic heterocyclic group.

The base-addition salts include, for example, alkali metal salts such as sodium salts, potassium salts; alkaline earth metal salts such as calcium salts, magnesium salts; ammonium salts; and organic amine salts such as trimethylamine salts, triethylamine salts, dicyclohexylamine salts, ethanolamine salts, diethanolamine salts, triethanolamine salts, procaine salts, N,N′-dibenzylethylenediamine salts.

The acid-addition salts include, for example, inorganic acid salts such as hydrochlorides, sulfates, nitrates, phosphates, perchlorates; organic acid salts such as maleates, fumarates, tartrates, citrates, ascorbates, trifluoroacetates; and sulfonates such as methanesulfonates, isethionates, benzenesulfonates, p-toluenesulfonates.

The term “pharmaceutically acceptable carrier or diluent” refers to excipients, (e.g., fats, beeswax, semi-solid and liquid polyols, natural or hydrogenated oils, etc.], water (e.g., distilled water, particularly distilled water for injection, etc.), physiological saline, alcohol (e.g., ethanol), glycerol, polyols, aqueous glucose solution, mannitol, plant oils, etc.), and additives (e.g., extending agent, disintegrating agent, binder, lubricant, wetting agent, stabilizer, emulsifier, dispersant, preservative, sweetener, colorant, seasoning agent or aromatizer, concentrating agent, diluent, buffer substance, solvent or solubilizing agent, chemical for achieving storage effect, salt for modifying osmotic pressure, coating agent or antioxidant, and the like).

Solid preparations can be prepared in the forms of tablet, capsule, granule and powder without any additives, or prepared using appropriate carriers (additives). Examples of such carriers (additives) may include saccharides such as lactose or glucose; starch of corn, wheat or rice; fatty acids such as stearic acid; inorganic salts such as magnesium metasilicate aluminate or anhydrous calcium phosphate; synthetic polymers such as polyvinylpyrrolidone or polyalkylene glycol; alcohols such as stearyl alcohol or benzyl alcohol; synthetic cellulose derivatives such as methylcellulose, carboxymethylcellulose, ethylcellulose or hydroxypropylmethylcellulose; and other conventionally used additives such as gelatin, talc, plant oil and gum arabic.

These solid preparations such as tablets, capsules, granules and powders may generally contain, for example, 0.1 to 100% by weight, and preferably 5 to 98% by weight, of the SYK inhibitor, based on the total weight of each preparation.

Liquid preparations are produced in the forms of suspension, syrup, injection and drip infusion (intravenous fluid) using appropriate additives that are conventionally used in liquid preparations, such as water, alcohol or a plant-derived oil such as soybean oil, peanut oil and sesame oil.

In particular, when the preparation is administered parenterally in a form of intramuscular injection, intravenous injection or subcutaneous injection, appropriate solvent or diluent may be exemplified by distilled water for injection, an aqueous solution of lidocaine hydrochloride (for intramuscular injection), physiological saline, aqueous glucose solution, ethanol, polyethylene glycol, propylene glycol, liquid for intravenous injection (e.g., an aqueous solution of citric acid, sodium citrate and the like) or an electrolytic solution (for intravenous drip infusion and intravenous injection), or a mixed solution thereof. Such injection may be in a form of a preliminarily dissolved solution, or in a form of powder per se or powder associated with a suitable carrier (additive) which is dissolved at the time of use. The injection liquid may contain, for example, 0.1 to 10% by weight of an active ingredient based on the total weight of each preparation.

Liquid preparations such as suspension or syrup for oral administration may contain, for example, 0.1 to 10% by weight of an active ingredient based on the total weight of each preparation.

Each preparation in the invention can be prepared by a person having ordinary skill in the art according to conventional methods or common techniques. For example, a preparation can be carried out, if the preparation is an oral preparation, for example, by mixing an appropriate amount of the compound of the invention with an appropriate amount of lactose and filling this mixture into hard gelatin capsules which are suitable for oral administration. On the other hand, preparation can be carried out, if the preparation containing the compound of the invention is an injection, for example, by mixing an appropriate amount of the compound of the invention with an appropriate amount of 0.9% physiological saline and filling this mixture in vials for injection.

The components of this invention may be administered to mammals, including humans, either alone or, in combination with pharmaceutically acceptable carriers, excipients or diluents, in a pharmaceutical composition, according to standard pharmaceutical practice. The components can be administered orally or parenterally, including the intravenous, intramuscular, intraperitoneal, subcutaneous, rectal and topical routes of administration.

Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved. Further information about suitable dosages is provided below.

The term “administration” and variants thereof (e.g., “administering” a compound) in reference to a component of the invention means introducing the component or a prodrug of the component into the system of the animal in need of treatment. When a component of the invention or prodrug thereof (e.g., the SYK inhibitor) is provided in combination with one or more other active agents, “administration” and its variants are each understood to include concurrent and sequential introduction of the component or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits a biological or medicinal response in a tissue, system, animal or human, that is being sought by a researcher, veterinarian, medical doctor or other clinician. This includes combination therapy involving the use of multiple therapeutic agents, such as a combined amount of a first and second treatment where the combined amount will achieve the desired biological response. The desired biological response is partial or total inhibition, delay or prevention of the progression of cancer including cancer metastasis; inhibition, delay or prevention of the recurrence of cancer including cancer metastasis; or the prevention of the onset or development of cancer (chemoprevention) in a mammal, for example a human.

A suitable amount of a SYK inhibitor is administered to a patient undergoing treatment for a B-cell lymphoma or leukemia. In an embodiment, a SYK inhibitor is administered in doses ranging from about 100 mg per day to 250 mg per day. In an embodiment of the invention, a SYK inhibitor is administered twice daily (BID), over the course of two and a half days, for a total of 5 doses. In another embodiment of the invention, a SYK inhibitor is administered once daily (QD) over the course of two days, for a total of 2 doses.

In an embodiment of the invention, a SYK inhibitor can be administered 5 times per week. In another embodiment of the invention, a SYK inhibitor can be administered 2 times per week.

In an embodiment of the invention, the SYK inhibitor administered in the present invention may be presented in unit dose forms containing a predetermined amount of active ingredient per unit dose. Such a unit may contain, for example, 5 μg to 1 g, preferably 1 mg to 700 mg, more preferably 5 mg to 100 mg of a SYK inhibitor, depending on the condition being treated, the route of administration and the age, weight and condition of the patient. Such unit doses may therefore be administered more than once a day. Preferred unit dosage compositions are those containing a daily dose or sub-dose (for administration more than once a day), as herein above recited, or an appropriate fraction thereof, of an active ingredient.

### Additional Anti-Cancer Agents

The SYK inhibitor administered in the methods of the instant invention is also useful in combination with additional therapeutic, chemotherapeutic and anti-cancer agents. Further combination with a SYK inhibitor of the instant invention with therapeutic, chemotherapeutic and anti-cancer agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such additional agents include the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs) and agents that interfere with cell cycle checkpoints. The mTOR inhibitor and αvβ3 integrin antagonist combination of the instant invention may be particularly useful when co-administered with radiation therapy.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, a-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell myosis, including alkylating agents, tumor necrosis factors, intercalators, hypoxia activatable compounds, microtubule inhibitors/microtubule-stabilizing agents, inhibitors of mitotic kinesins, histone deacetylase inhibitors, inhibitors of kinases involved in mitotic progression, inhibitors of kinases involved in growth factor and cytokine signal transduction pathways, antimetabolites, biological response modifiers, hormonal/anti-hormonal therapeutic agents, haematopoietic growth factors, monoclonal antibody targeted therapeutic agents, topoisomerase inhibitors, proteosome inhibitors, ubiquitin ligase inhibitors, and aurora kinase inhibitors.

Examples of cytotoxic/cytostatic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, 4-demethoxy-3-deamino-3-aziridinyl-4-methylsulphonyl-daunorubicin (see WO 00/50032), Raf kinase inhibitors (such as Bay43-9006) and mTOR inhibitors, such as ridaforolimus, everolimus, temsirolimus, sirolimus or a rapamycin-analog.

An example of a hypoxia activated compound is tirapazamine.

Examples of proteosome inhibitors include but are not limited to lactacystin and MLN-341 (Velcade).

Examples of microtubule inhibitors/microtubule-stabilizing agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797. In an embodiment the epothilones are not included in the microtubule inhibitors/microtubule-stabilising agents.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in Publications WO 2003/039460, WO 2003/050064, WO 2003/050122, WO 2003/049527, WO 2003/049679, WO 2003/049678, WO 2004/039774, WO 2003/079973, WO 2003/099211, WO 2003/105855, WO 2003/106417, WO 2004/037171, WO 2004/058148, WO 2004/058700, WO 2004/126699, WO 2005/018638, WO 2005/019206, WO 2005/019205, WO 2005/018547, WO 2005/017190, US 2005/0176776. In an embodiment inhibitors of mitotic kinesins include, but are not limited to, inhibitors of KSP, inhibitors of MKLP1, inhibitors of CENP-E, inhibitors of MCAK, and inhibitors of Rab6-KIFL.

Examples of “histone deacetylase inhibitors” include, but are not limited to, SAHA, TSA, oxamflatin, PXD101, MG98 and scriptaid. Further reference to other histone deacetylase inhibitors may be found in the following manuscript; Miller, T. A., et al., J. Med. Chem., 2003, 46(24):5097-5116.

“Inhibitors of kinases involved in mitotic progression” include, but are not limited to, inhibitors of aurora kinase, inhibitors of Polo-like kinases (PLK; in particular inhibitors of PLK-1), inhibitors of bub-1 and inhibitors of bub-R1. An example of an “aurora kinase inhibitor” is VX-680.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, 3-aminopyridine-2-carboxaldehyde thiosemicarbazone, and trastuzumab.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include, but are not limited to, lovastatin (MEVACOR®; see U.S. Pat. Nos. 4,231,938, 4,294,926 and 4,319,039), simvastatin (ZOCORθ; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOLθ; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, Cholesterol Lowering Drugs, Chemistry & Industry, 1996, pp. 85-89, and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis, see, European J. of Cancer, 1999, 35(9):1394-1401.

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs), like aspirin and ibuprofen, as well as selective cyclooxy-genase-2 inhibitors like celecoxib and rofecoxib (PNAS, 1992, 89:7384; JNCI, 1982, 69:475; Arch. Opthalmol., 1990, 108:573; Anat. Rec., 1994, 238:68; FEBS Letters, 1995, 372:83; Clin, Orthop., 1995, 313:76; J. Mol. Endocrinol., 1996, 16:07; Jpn. J. Pharmacol., 1997, 75:105; Cancer Res., 1997, 57:1625; Cell, 1998, 93:705; Intl. J. Mol. Med., 1998, 2:715; J. Biol. Chem., 1999. 274:9116), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see, Fernandez, et al., J. Lab. Clin. Med., 1985, 105:141-145), and antibodies to VEGF (see, Nature Biotechnology, 1999, 17:963-968); Kim, et al., Nature, 1993, 362:841-844; WO 2000/44777; and WO 2000/61186).

Other therapeutic agents that modulate or inhibit angiogenesis and may also be used in combination with the SYK inhibitor of the instant invention, include agents that modulate or inhibit the coagulation and fibrinolysis systems (see, review in Clin. Chem. La. Med., 2000, 38:679-692). Examples of such agents that modulate or inhibit the coagulation and fibrinolysis pathways include, but are not limited to, heparin (see, Thromb. Haemost., 1998, 80:10-23), low molecular weight heparins and carboxypeptidase U inhibitors (also known as, inhibitors of active thrombin activatable fibrinolysis inhibitor [TAFIa]) (see, Thrombosis Res., 2001, 101:329-354). TAFIa inhibitors have been described in PCT International Publication WO 2003/013526. “Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, and CHK1 kinases and cdk and cdc kinase inhibitors and are specifically exemplified by 7-hydroxy-staurosporin, flavopiridol, CYC202 (Cyclacel) and BMS-387032.

“Agents that interfere with receptor tyrosine kinases (RTKs)” refer to compounds that inhibit RTKs and therefore mechanisms involved in oncogenesis and tumor progression. Such agents include inhibitors of c-Kit, Eph, PDGF, F1t3 and c-Met. Further agents include inhibitors of RTKs as described by Bume-Jensen and Hunter, Nature, 2001, 411:355-365.

“Inhibitors of cell proliferation and survival signaling pathway” refer to compounds that inhibit signal transduction cascades downstream of cell surface receptors. Such agents include inhibitors of serine/threonine kinases (including but not limited to inhibitors of Akt such as described in WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344, US 2005/029941, US 2005/44294, US 2005/43361, WO 2006/135627, WO 2006/091395, WO 2006/110638), inhibitors of Raf kinase (for example BAY-43-9006), inhibitors of MEK (for example CI-1040 and PD-098059), inhibitors of mTOR (for example Wyeth CCI-779), and inhibitors of PI3K (for example LY294002).

Specific anti-IGF-1R antibodies include, but are not limited to, dalotuzumab, figitumumab, cixutumumab, SHC 717454, Roche R1507, EM164 or Amgen AMG479.

As described above, the combinations with NSAID's are directed to the use of NSAID's which are potent COX-2 inhibiting agents. For purposes of this specification an NSAID is potent if it possesses an IC₅₀ for the inhibition of COX-2 of 1 μM or less as measured by cell or microsomal assays.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC50 for COX-1 evaluated by cell or microsomal assays. Such compounds include, but are not limited to, those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142, U.S. Pat. No. 5,380,738, U.S. Pat. No. 5,393,790, U.S. Pat. No. 5,466,823,U.S. Pat. No. 5,633,272, and U.S. Pat. No. 5,932,598.

Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5-pyridinyl)pyridine, or a pharmaceutically acceptable salt thereof.

Compounds that have been described as specific inhibitors of COX-2 and are therefore useful in the present invention include, but are not limited to, the following: parecoxib, BEXTRA® and CELEBREX® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yhchloroacetyl)carbamate, acetyldinanaline, 5-amino-1-[[3,5-dichloro-4-(4-chlorobenzoyl)phenyl]methyl]-1H-1,2,3-triazole-4-carboxamide,CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the α_{V}β₃ integrin, to compounds which selectively antagonize, inhibit or counteract binding of a physiological ligand to the αvβ5 integrin, to compounds which antagonize, inhibit or counteract binding of a physiological ligand to both the α_{V}β₃ integrin and the α_{V}β₅ integrin, and to compounds which antagonize, inhibit or counteract the activity of the particular integrin(s) expressed on capillary endothelial cells. The term also refers to antagonists of the α_{V}β₆, α_{V}β₈, α₁β₁, α₂β₁, α₅β₁, α₆β₁, and α₆β₄ integrins. The term also refers to antagonists of any combination of α_{V}β₃, α_{V}β₅, α_{V}β₆, α_{V}β₈, α₁β₁, α₂β₁, α₅β₁, α₆β₁, and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3 ‘,2’,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD 121974.

Combinations with compounds other than anti-cancer compounds are also encompassed in the instant methods. For example, combinations of the mTOR inhibitor and αvβ3 integrin antagonist combination of the instant invention with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see, J. Cardiovasc. Pharmacol., 1998, 31:909-913; J. Biol. Chem., 1999, 274:9116-9121; Invest. Ophthalmol Vis. Sci., 2000, 41:2309-2317). More recently, PPAR-γ agonists have been shown to inhibit the angiogenic response to VEGF in vitro; both troglitazone and rosiglitazone maleate inhibit the development of retinal neovascularization in mice (Arch. Ophthamol., 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy) phenoxy)propoxy)-2-ethylchromane-2-carboxylic acid (disclosed in U.S. Ser. No. 60/235,708 and 60/244,697).

Another embodiment of the instant invention is the use of the SYK inhibitor in combination with gene therapy for the treatment of cancer. For an overview of genetic strategies to treat cancer, see, Hall, et al., Am. J. Hum. Genet., 1997, 61:785-789 and Kufe, et al., Cancer Medicine, 5th Ed, B. C. Decker, Hamilton, 2000, pp 876-889. Gene therapy can be used to deliver any tumor suppressing gene. Examples of such genes include, but are not limited to, p53, which can be delivered via recombinant virus-mediated gene transfer (see, U.S. Pat. No. 6,069,134), a uPA/uPAR antagonist (Gene Therapy, 1998, 5(8):1105-13), and interferon gamma (J. Immunol., 2000, 164:217-222).

The SYK inhibitor administered in the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

The SYK inhibitor administered in the instant invention may be employed in conjunction with anti-emetic agents to treat nausea or emesis, including acute, delayed, late-phase, and anticipatory emesis, alone or with radiation therapy. For the prevention or treatment of emesis, the SYK inhibitor may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as, the phenothiazines (for example, prochlorperazine, fluphenazine, thioridazine and mesoridazine), metoclopramide or dronabinol. In another embodiment, conjunctive therapy with an anti-emesis agent selected from a neurokinin-1 receptor antagonist, a 5HT3 receptor antagonist and a corticosteroid is disclosed for the treatment or prevention of emesis that may result upon administration of the SYK inhibitor.

Neurokinin-1 receptor antagonists of use in conjunction with the SYK inhibitor used in the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913,0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications.

In an embodiment, the neurokinin-1 receptor antagonist for use in conjunction with the SYK inhibitor administered in the instant invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

The SYK inhibitor administered in the instant invention may also be administered with an agent useful in the treatment of anemia. Such an anemia treatment agent is, for example, a continuous erythropoiesis receptor activator (such as, Epoetin alfa).

The SYK inhibitor administered in the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

The SYK inhibitor administered in the instant invention may also be administered with an immunologic-enhancing drug, such as levamisole, isoprinosine and Zadaxin.

The SYK inhibitor administered in the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

The SYK inhibitor administered in the instant invention may also be useful for treating or preventing breast cancer in combination with aromatase inhibitors. Examples of aromatase inhibitors include but are not limited to: anastrozole, letrozole and exemestane.

The SYK inhibitor administered in the instant invention may also be useful for treating or preventing cancer in combination with siRNA therapeutics.

The SYK inhibitor administered in the instant invention may also be administered in combination with γ-secretase inhibitors and/or inhibitors of NOTCH signaling. Such inhibitors include compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, U.S. Ser. No. 10/957,251, WO 2004/089911, WO 02/081435, WO 02/081433, WO 03/018543, WO 2004/031137, WO 2004/031139, WO 2004/031138, WO 2004/101538, WO 2004/101539 and WO 02/47671 (including LY-450139).

The SYK inhibitor administered in the instant invention may also be useful for treating or preventing cancer in combination with inhibitors of Akt. Such inhibitors include compounds described in, but not limited to, the following publications: WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344, US 2005/029941, US 2005/44294, US 2005/43361, WO 2006/135627, WO 2006091395, WO 2006/110638).

The SYK inhibitor administered in the instant invention may also be useful for treating or preventing cancer in combination with PARP inhibitors.

Radiation therapy itself means an ordinary method in the field of treatment of cancer. For radiation therapy, employable are various radiations such as X-ray, γ-ray, neutron ray, electron beam, proton beam; and radiation sources. In a most popular radiation therapy, a linear accelerator is used for irradiation with external radiations, γ-ray.

The SYK inhibitor administered in the instant invention may also be useful for treating cancer in further combination with the following therapeutic agents: abarelix (Plenaxis Depot®); abiraterone acetate (Zytiga®); (Actiq®); aldesleukin (Prokine®); Aldesleukin (Proleukin®); Alemtuzumab (Campath®); alfuzosin HCl (UroXatral®); alitretinoin (Panretin®); allopurinol (Zyloprim®); altretamine (Hexalen®); amifostine (Ethyol®); anastrozole (Arimidex®); (Anzemet®); (Anexsia®); aprepitant (Emend®); arsenic trioxide (Trisenox®); asparaginase (Elspar®); azacitidine (Vidaza®); bendamustine hydrochloride (Treanda®); bevacuzimab (Avastin®); bexarotene capsules (Targretin®); bexarotene gel (Targretin®); bleomycin (Blenoxane®); bortezomib (Velcade®); (Brofenac®); busulfan intravenous (Busulflex®); busulfan oral (Myleran®); cabazitaxel (Jevtana®); calusterone (Methosarb); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cinacalcet (Sensipar®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); dasatinib (Sprycel); daunorubicin liposomal (DanuoXome); daunorubicin, daunomycin (Daunorubicin®); daunorubicin, daunomycin (Cerubidine®); decitabine (Dacogen®); degarelix (Degarelix®); Denileukin diftitox (Ontak®); denosumab (Xgeva®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); Elliott's B Solution (Elliott's B Solution®); epirubicin (Ellence®); Epoetin alfa (Epogen®); eribulin mesylate (Halaven®); erlotinib (Tarceva®); estramustine (Emcyt®); etoposide phosphate (Etopophos®); etoposide, VP-16 (Vepesid®); everolimus (Afinitor®); exemestane (Aromasin®); fentanyl buccal (Onsolis®); fentanyl citrate (Fentora®); fentanyl sublingual tablets (Abstral®); Filgrastim (Neupogen®); floxuridine (intraarterial) (FUDR®); fludarabine (Fludara®); fluorouracil, 5-FU (Adrucil®); flutamide (Eulexin®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); granisetron (Kytril Solution®) (Sancuso®); histrelin acetate (Histrelin Implant®); human papillomavirus bivalent vaccine (Cervarix®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); ipilimumab (Yervoy®); irinotecan (Camptosar®); (Kadian®); ixabepilone (Ixempra®); lapatinib (Tykerb®); lenalidomide (Revlimid®); letrozole (Femara®); leucovorin (Wellcovorin®, Leucovorin®); Leuprolide Acetate (Eligard®); (Lupron Depot®); (Viadur®); levamisole (Ergamisol®); levoleucovorin (Fusilev®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitomycin C (Mitozytrex®); mitotane (Lysodren®); mitoxantrone (Novantrone®); nandrolone phenpropionate (Durabolin-50®); nelarabine (Arranon); nilotinib hydrochloride monohydrate (Tasigna®); Nofetumomab (Verluma®); ofatumumab (Arzerra®); ondansetron (Zuplenz®); Oprelvekin (Neumega®); (Neupogen®); oxaliplatin (Eloxatin®); paclitaxel (Paxene®); paclitaxel (Taxol®); paclitaxel protein-bound particles (Abraxane®); palifermin (Kepivance®); palonosetron (Aloxi®); pamidronate (Aredia®); panitumumab (Vectibix®); pazopanib (Votrient®); pegademase (Adagen (Pegademase Bovine)®); pegaspargase (Oncaspar®); Pegfilgrastim (Neulasta®); peginterferon alfa-2B (Sylatron®); pemetrexed disodium (Alimta®); pentostatin (Nipent®); pipobroman (Vercyte®); plerixafor injection (Mozobil®); plicamycin, mithramycin (Mithracin®); porfimer sodium (Photofrin®); pralatrexate injection (Folotyn®); procarbazine (Matulane®); (Quadramet®); quadrivalent human papillomavirus (types 6, 11, 16, 18) recombinant vaccine (Gardasil®); quinacrine (Atabrine®); raloxifene hydrochloride (Evista®); Rasburicase (Elitek®); Rituximab (Rituxan®); romidepsin (Istodax); sargramostim (Leukine®); Sargramostim (Prokine®); secretin (SecreFlo); sipuleucel-T (Provenge®); sorafenib (Nexavar®); streptozocin (Zanosar®); sunitinib maleate (Sutent®); talc (Sclerosol®); tamoxifen (Nolvadex®); temozolomide (Temodar®); temsirolimus (Torisel®); teniposide, VM-26 (Vumon®); (Temodar®); testolactone (Teslac®); thalidomide (Thalomid®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); (Trelstar LA®); tretinoin, ATRA (Vesanoid®); triptorelin pamoate (Trelstar Depot®); (UltraJect®); Uracil Mustard (Uracil Mustard Capsules®); valrubicin (Valstar®); vandetanib (Vandetanib®); vinblastine (Velban®); vincristine (Oncovin®); vinorelbine (Navelbine®); vorinostat (Zolinza); (Zofran ODT); and zoledronate (Zometa®).

### EXAMPLES

### Example 1

### General Material and Methods

### A. Cell Culture

Diffuse, large B-cell lymphoma (DLBCL) cell lines DHL4, Farage, LY18, LY19, Pfeiffer, Toledo, U2932 and Wsu-NHL cells were cultured in RPIM 1640 medium (Gibco® cell culture, Invitrogen, Carlsbad, Calif.) with 10% FBS with glucose. DHL6, DHL8, DHL10, DB and LY1 cells were cultured with RPMI medium (Gibco® cell culture, Invitrogen, Carlsbad, Calif.) with 20% FBS with glucose. LY3, LY4, LY7 and LY10 were cultured in IMDM medium (Gibco® medium, Invitrogen, Carlsbad, Calif.) with 20% FBS and glucose. All cell lines were maintained at 37 degrees, 5% CO₂, in an incubator. All cells were mycoplasma tested negative.

### B. Cell Viability Assay

Seventeen DLBCL cell lines were treated with the SYK-1 inhibitor (WO 2012/154519) at 10, 3, 1, 0.3, 0.1, 0.03, 0.01, 0.003, 0.001 and DMSO alone for 5 days. Cells were lysed and cell viability was determined by measuring ATP activity using CellTiter-Glo® (Promega, Madison, Wis.).

### C. RNA Extraction and Microarray Data Processing

Total RNA was extracted from cell lines after homogenization in 750 μl of 100% Trizol. 100% Chloroform was added to the lysate in a 1:5 ratio to facilitate separation of the organic and aqueous components. The aqueous supernatant was purified using the SV 96 Total RNA kit (Promega, Madison, Wis.), incorporating a DNase treatment during the procedure. Isolated total RNA samples were then assayed for quality (Bioanalyzer, Agilent Technologies, Santa Clara, Calif.) and yield (RiboGreen® RNA Assay kit, Life Technologies, Carlsbad, Calif.) metrics prior to amplification. Samples were then amplified using the Ovation® Whole Blood protocol (NuGEN Technologies, San Carlos, Calif.) and hybridized to Rosetta/Merck Human RSTA Custom 1.0 microarrays (GEO accession number GPL6793, Affymetrix, Santa Clara, Calif.). Microarray data was processed using RMA normalization as implemented in R Bioconductor.

### D. Derivation of Meta-Genes and Predictive Signature

Applicants define a meta-gene as a set of transcripts showing significant correlation in expression levels across tumor samples. Meta-gene expression is calculated as the geometric average expression intensity of the individual genes making up the meta-gene. Meta-genes were derived from previously published, pre-treatment tumor biopsy microarray data from three separate studies (Lenz et al., 2008, N. Engl. J. Med., 359(22):2313-2323; Compagno et al. 2009, Nature, 459(7247):717-721; Shaknovich et al., 2010, Blood, 116(20):e81-89).

DLBCL microarray samples from these three studies were combined and normalized by robust multiarray averaging. Batch effects were estimated by fitting a linear model to the expression data using limma. Batch effects for each gene were subtracted prior to further analysis. Meta-genes were derived by clustering the 5,000 most variable mRNA transcripts into groups with highly correlated expression across the 596 patient samples.

Clustering was performed on the gene-gene correlation matrix using the apcluster package in R with default settings (open source software, R Project for Statistical Computing, Institute for Statistics and Mathematics, WU (Wirtschaftsuniversität Wein, Vienna University of Economics and Business, Vienna, Austria). This yielded a total of 507 clusters ranging from a single transcript in size to 83 transcripts, with a median size of five transcripts.

Applicants then evaluated the coherence of these clusters in the seventeen DLBCL cell lines by comparing the median gene-gene correlation observed within a cluster to an empirical distribution obtained from 1,000 randomly generated clusters of the same size. Only the 324 clusters with significantly higher median gene-gene correlation than expected by chance (p<0.01) were carried forward as meta-genes. The Spearman correlation between meta-gene expression and cell viability was calculated.

### E. Flow Cytometric Analysis of Surface CD86

Surface CD86 expression levels were measured using PE conjugated antibody against human CD86 (BD, Becton, Dickinson, and Company, Franklin Lakes, N.J.) and its isotype control antibody (BD, Becton, Dickinson, and Company, Franklin Lakes, N.J.). The intensity of fluorescence was detected by BD™ LSR II Flow Cytometry (BD Bioscience, Franklin Lakes, N.J.) and analyzed with Deva software (Roche NimbleGen, Madison, Wis.).

### F. Immunohistochemistry

Tissue Micro Array (#LYM1021, Pantomics, Richmond, Calif.) from 90 DLBCL patient samples were stained with an antibody against human CD20, a B-cell marker used to verify the presence of DLBCL in the sample (Ventana Medical Systems, Oro Valley, Ariz.) and CD86 (Santa Cruz Biotechnology, Santa Cruz, Calif.) using Ventana Discovery Ultra (Ventana Medical Systems, Oro Valley, Ariz.). CD86 expression was measured only in the portion of the sample that was positive for CD20. H-scores were determined using Aperio's algorithm for CD20 and CD86 staining (Leica Microsystems).

### Example 2

### Determination of SYK Inhibition

Compounds to be used as SYK inhibitors in the inventive methods herein may be evaluated for SYK inhibition using a homogeneous time-resolved fluorescence (HTRF) assay for the recombinant human SYK enzyme as follows.

A recombinant GST-hSYK fusion protein was used to measure potency of compounds to inhibit human SYK activity. The recombinant human GST-Syk (Carna Biosciences, #08-176, Kobe, Japan) (5 pM final concentration) was incubated with various concentrations of the inhibitor diluted in DMSO (0.1% final concentration) for 10 minutes at room temperature in 15 mM Tris-HCl (pH 7.5), 0.01% tween 20, 2 mM DTT in a 384 well plate format.

To initiate the reaction, the biotinylated substrate peptide (250 nM final concentration) that contains the phosphorylation site for SYK was added with magnesium (5 mM final concentration) and ATP (25 μM final concentration). Final volume of the reaction was 10 iut. Phosphorylation of the peptide was allowed to proceed for 45 minutes at room temperature. To quench the reaction and detect the phosphorylated product, 2 nM of a Europium-anti-phosphotyrosine antibody (Perkin Elmer, #AD0161, Waltham, Mass.) and 70 nM SA-APC (Perkin-Elmer, #CR130-100, Waltham, Mass.) were added together in 15 mM Tris pH 7.5, 40 mM EDTA, 0.01% Tween 20. Final volume of the quenching solution was 10 μL.

The resulting HTRF signal was measured after 30 minutes on an EnVision® Multilable Plate Reader (Perkin-Elmer, Waltham, Mass.) using a time-resolved fluorescence protocol. The IC₅₀ was determined following a 10-dose titration (10 μM to 0.508 nM) and a four parameter logistic curve fitting using the Merck Assay Data Analyzer. The rhSYK activity (IC₅₀) was expressed as follows: “+++” for values of 100 nM or less, “++” for values between 100 and 1000 nM), and “+” for values between 1 and 10 μM.

### Example 3

### DLBCL Cell Line Gene Expression Predicts SYK Inhibitor Sensitivity

To evaluate whether pre-treatment mRNA expression level correlated with an in vitro response to SYK inhibition, Applicants assayed the sensitivity of seventeen DLBCL cell lines to the SYK inhibitor, SYK-1, and measured genome-wide mRNA expression levels in these same cell lines using Affymetrix microarrays. The Spearman correlation between the SYK-1 sensitivity and the expression of 324 pre-specified meta-genes was assessed according to the methods of Example 1. Meta-genes with a statistically significant Spearman correlation coefficient and a plausible biological link to the SYK-mediated pathways were combined into a gene signature, i.e., SYK gene signature. The Meta-Genes, and the genes comprising each Meta-gene, comprising the SYK gene signature are provided in Table 1.

TABLE 1

| | | | Genes Comprising Meta-Gene | |
|---|---|---|---|---|
| | | | NCBI Transcript Number | |
| Meta-gene | r_{DLBCL} | R_{Validation} | mRNA | Protein |
| BCR Signaling | 0.52 | 0.64 | CLECL1 (NM_172004) | CLECL1 (NP_742001) |
| | | | ZNF107 (NM_001013746) | ZNF107 (NP_001013768) |
| | | | CD72 (NM_001782) | CD72 (NP_001773) |
| | | | BLNK (NM_013314) | BLNK (NP_037446) |
| | | | HSH2D (NM_032855) | HSH2D (NP_116244) |
| | | | TMEM154 (NM_15680) | TMEM154 (NP_689893) |
| | | | MCOLN2 (NM_153259) | MCOLN2 (NP_694991) |
| | | | C1orf186 (NM_001007544) | C1orf186 (NP_001007545) |
| | | | C7orf23 (NM_024315) | C7orf23 (NP_077291) |
| | | | LYN (NM_ 02350) | LYN (NP_002341) |
| | | | SP140 (NM_007237) | SP140 (NP_009168) |
| | | | SPIB (NM_003121) | SPIB (NP_003112) |
| NFκB Activation | 0.54 | 0.40 | LAT2 (NM_032463) | LAT (NP_115852) |
| | | | TP63 (NM_003722) | TP63 (NP_003713) |
| | | | DNAJC5B (NM_033105) | DNAJC5B (NP_149096) |
| | | | SNX8 (NM_013321) | SNX8 (NP_037453) |
| | | | LY75 (NM_002349) | LY75 (NP_002340) |
| | | | CD40 (NM_152854) | CD40 (NP_690593) |
| | | | RHOF (NM_019034) | RHOF (NP_061907) |
| | | | TSPAN33 (NM_178562) | TSPAN33 (NP_848657) |
| CD86 | 0.44 | 0.47 | CD86 (NM_175862) | CD86 (NP_787058) |
| SORL1/MYO1E | 0.62 | 0.47 | SORL1 (NM_003105) | SORL1 (NP_003096) |
| | | | MYO1E (NM_004998) | MYO1E (NP_004989) |

For each cell line Applicants calculated a signature score by averaging the log expression of all genes in the signature for that cell line. The coefficient of determination between this score and the log IC₅₀ of cell viability dose response in the seventeen DLBCL cell line panel was 0.47.

To validate the ability of this gene signature to predict a response to SYK inhibition, Applicants measured the pre-treatment gene expression levels for all genes comprising the signature in a panel of 46 B-cell lines. Although this panel did not include additional DLBCL cell lines, Applicants reasoned that the dysregulated pathways, that is, pathways that are either up- or down-regulated in malignant cells as compared to normal B-cells, mediated by SYK activity may be shared among multiple B-cell malignancies. FIG. 2 is a graphic illustration of the relationship between SYK inhibition sensitivity and signature score in this panel.

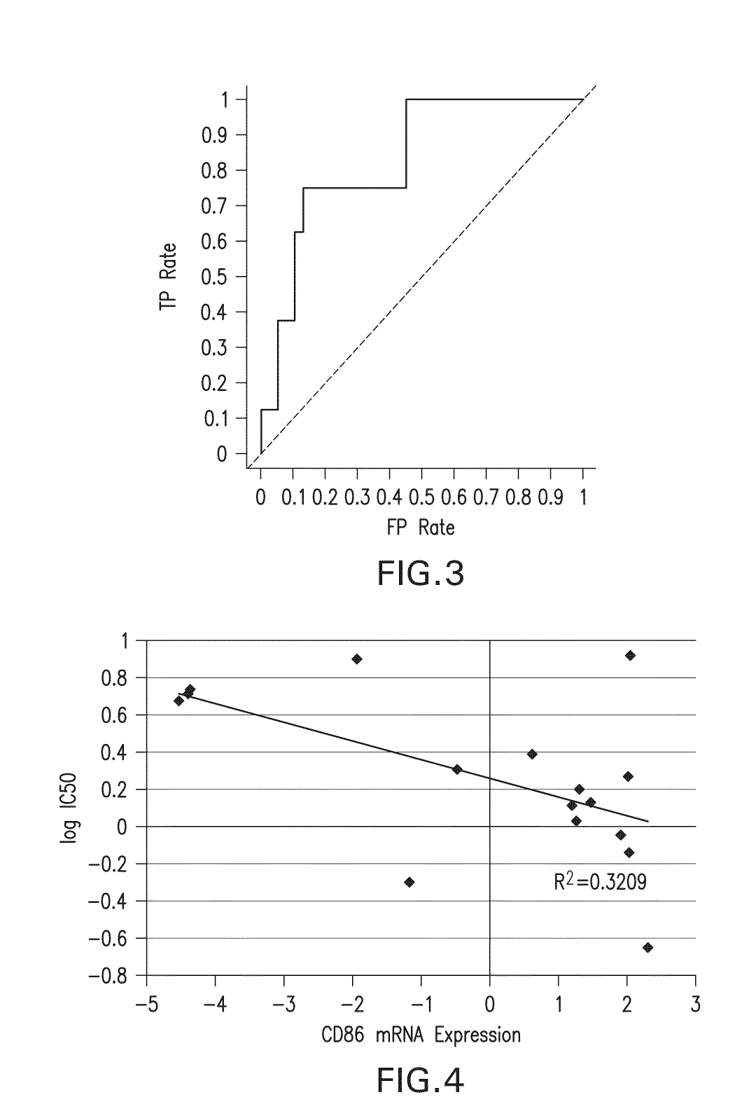

FIG. 3 is a graphic illustration of the receiver operating characteristic (ROC) for the gene signature applied to this 46 gene panel. Responders were defined as cell lines with equal or greater sensitivity to SYK inhibition than SUDHL4 (a known sensitive cell line). The area under the ROC curve is 0.83 indicating that the signature has good predictive value in this validation set.

Among the genes comprising the SYK signature, several genes appeared to have a particularly strong biological correlation to known SYK-mediated pathways and B-cell receptor signaling. As shown in Table 2, the listed genes have previously been described as having a role in B-cell receptor signaling or SYK-mediated pathways.

TABLE 2

| Gene | Description | Refs |
|---|---|---|
| CLECL1 | B-cell surface expressed co- | Ryan et al., 2002, J. |
| | stimulatory molecule | Immunol., 169(10): 5638-5648 |
| CD72 | Regulator of B-cell receptor | Baba et al., 2005, Eur. J. |
| | signaling | Immunol., 35(5): 1634-1642 |
| BLNK | B-cell receptor signaling | Harwood and Batista, 2009, |
| | mediator | Ann. Rev. Immunol., 28: |
| | | 185-210 |
| HSH2D | Negative regulator of | Herrin et al., 2005. J. Biol. |
| | apoptosis upon B-cell | Chem., 280(5): 3507-3515 |
| | receptor ligation | |
| MCOLN2 | BTK transcriptional | Lindvall et al., 2005, Cell |
| | target | Immunol., 235(1): 46-55 |
| LYN | Activated during BCR | Harwood and Batista, 2010, |
| | signaling, component of | Ann. Rev. Immunol., 28: |
| | BCR signaling complex | 185-210 |
| SPIB | Transcription factor re- | Garrett-Sinha et al., 1999, |
| | quired for normal BCR | Immunity, 10(4): 399-408 |
| | signaling. | |
| CD40 | Can augment BCR signal- | Ying et al., 2011, |
| | ing via SYK-mediated | Immunobiology, 216(5): |
| | mechanism. | 566-570 |
| MYO1E | BTK transcriptional | Lindvall et al., 2005, Cell |
| | target | Immunol., 235(1): 46-55 |
| CD86 | Surface expression up- | Holodick et al., 2009, Mol. |
| | regulated upon BCR | Immun., 46(15): 3029-3036 |
| | ligation, and downregu- | |
| | lated by SYK inhibition. | |

In reviewing the genes comprising the gene signature, CD86 was of particular interest in that higher mRNA expression of CD86 was correlated with SYK inhibitor sensitivity, CD86 expression on the surface of B-cells is up-regulated by B-cell receptor ligation, and SYK inhibition has been reported to down-regulate CD86 expression in B-cells. FIG. 4 is a graphic illustration of the relationship between CD86 mRNA expression and SYK-1 inhibitor sensitivity in the seventeen DLBCL cell line panel.

### Example 4

### DLBCL Sensitivity to SYK-1 Correlates to the Level of Cell Surface CD86

Following of discovery of high CD86 mRNA expression in SYK-1 sensitive DLBCL lines, Applicants evaluated whether SYK-1 sensitivity also correlates to the protein level of CD86. Cells from the seventeen DLBCL cell lines were taken simultaneously to detect the level of cell surface CD86 by use of a fluorescent labeled antibody against CD86. The mean fluorescence intensity of CD86 staining on all cell lines was compared to SYK-1 sensitivity.

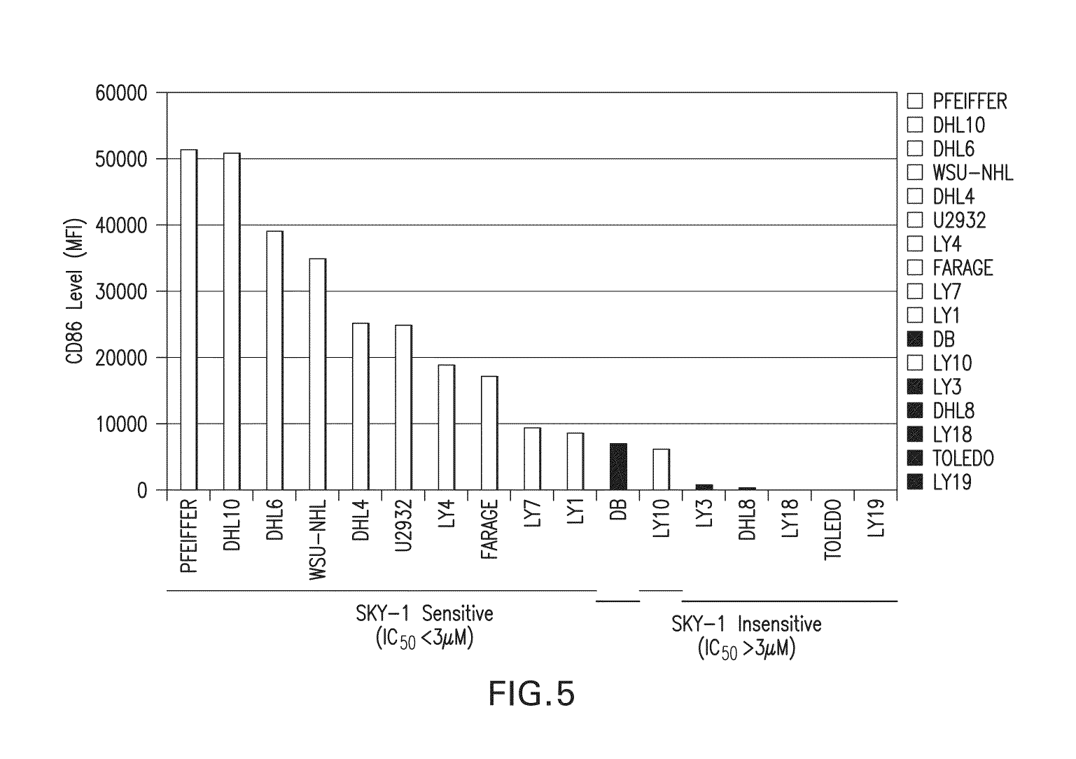

As shown in FIG. 5, cell lines expressing high levels of CD86 were sensitive to the SYK inhibitor, SYK-1, while cells with low or no expression of CD86 were resistant to inhibition with the SYK inhibitor, SYK-1. This finding suggested that CD86 could potentially be used as a predictive biomarker to identify patients diagnosed with a B-cell lymphoma or leukemia that were sensitive to treatment with a SYK inhibitor.

### Example 5

### CD86 as a Predictive Biomarker for Patient Selection

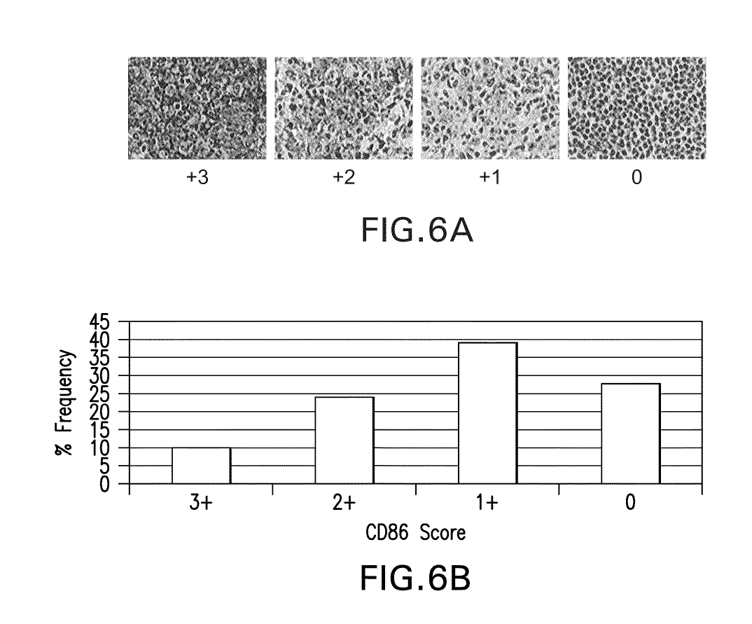

To validate the use of CD86 as a predictive biomarker, Applicants used a known immunohistochemical specific antibody to CD86 against tumor biopsy samples. To establish the CD86 positive/negative populations among DLBCL patients, a tissue microarray of 90 DLBCL patient samples was obtained. Excluding the CD20 negative area of the tumor biopsy samples, the samples were scored for the level of CD86 expression (FIG. 6A). As shown in FIG. 6B, 28% of the tumor biopsy samples were CD86 negative. This finding suggested that the exclusion of CD86 negative patients would enrich the population of DLBCL patients likely to respond to treatment with a SYK inhibitor. Alternatively stated, this suggested that patients, diagnosed with a B-cell lymphoma or leukemia, were more likely to respond to treatment with a SYK inhibitor if they were positive for CD86.

1. A method for treating a patient diagnosed with a B-cell lymphoma or leukemia with a SYK inhibitor comprising the steps of:
(a) selecting a patient for treatment with a SYK inhibitor, wherein a malignant B-cell containing biological sample of the patient has elevated CD86 expression; and
(b) administering a therapeutically effective amount of the SYK inhibitor to the selected patient.

2. The method of claim 1, comprising the steps of:
(a) measuring the expression level of CD86 in the biological sample from said patient;
(b) determining whether the CD86 expression level in said patient sample is above or below the level of a control sample;
(c) selecting said patient for treatment with a SYK inhibitor, wherein the level of the CD86 expression from said patient sample is at or above that of the control sample; and
(d) administering a therapeutically effective amount of the SYK inhibitor to the selected patient.

3. The method according to claim 1, wherein said control sample is a malignant B-cell containing biological sample obtained from one or more subjects diagnosed with B-cell lymphoma or leukemia, but do not respond to treatment with a SYK inhibitor.

4. A method for treating a patient diagnosed with a B-cell lymphoma or leukemia, in which a malignant B-cell containing biological sample of the patient has elevated expression levels of CD86, comprising the step of: administering a therapeutically effective amount of a SYK inhibitor to the patient.

5. The method according to claim 4, wherein the elevated expression levels of CD86 is in comparison to a reference derived from the CD86 expression level in a malignant B-cell containing biological sample of one or more subjects diagnosed with B-cell lymphoma or leukemia but do not respond to treatment with a SYK inhibitor.

6. The method according to claim 4, wherein the mRNA or protein expression level of CD86 is measured.

7. The method according to claim 4, wherein said B-cell lymphoma or leukemia is selected from the group consisting of acute leukemia, chronic lymphatic leukemia, chronic myelocytic leukemia, and non-Hodgkin's lymphoma.

8. The method according to claim 4, wherein the patient is diagnosed with diffuse large B-cell lymphoma.

9. A method for treating a B-cell lymphoma or leukemia patient, comprising the step of administering a therapeutically effective amount of a SYK inhibitor to the patient, wherein a malignant B-cell containing biological sample of said patient is characterized by elevated expression of CD86.

10. The method according to claim 9, wherein the elevated expression levels of CD86 is in comparison to a reference derived from the CD86 expression level in a malignant B-cell containing biological sample of one or more subjects diagnosed with B-cell lymphoma or leukemia but do not respond to treatment with a SYK inhibitor.

11. The method according to claim 9, wherein the mRNA or protein expression level of CD86 is measured.

12. The method according to claim 9, wherein said B-cell lymphoma or leukemia is selected from the group consisting of acute leukemia, chronic lymphatic leukemia, chronic myelocytic leukemia, and non-Hodgkin's lymphoma.

13. The method according to claim 9, wherein the patient is diagnosed with diffuse large B-cell lymphoma.

14. A kit to identify a B-cell lymphoma or leukemia patient sensitive to treatment with a SYK inhibitor comprising a detection agent capable of detecting the expression product of CD86 in a biological test sample.
